(19) United States
(12) Patent Application Publication
(10) Pub. No.: US 2026/0325797 A1
(43) Pub. Date: Oct. 22, 2026

(54) VALVED CATHETER DEVICES AND METHOD OF ASSEMBLING SUCH A DEVICE

(71) Applicant: B. Braun Melsungen AG, Melsungen (DE)

(72) Inventor: Hui Kuun Teoh, Penang (MY)

(73) Assignee: B. Braun Melsungen AG, Melsungen (DE)

(21) Appl. No.: 18/880,948

(22) PCT Filed: Jul. 6, 2023
(86) PCT No.: PCT/EP2023/068726

§ 371 Date: Jan. 3, 2025

Related U.S. Application Data
(60) Provisional application No. 63/367,780, filed on Jul. 6, 2022.

(51) Int. Cl. A61M 25/06; A61M 25/00; A61M 39/00; A61M 39/24

(52) U.S. Cl. CPC A61M 25/0606; A61M 25/0097; A61M 25/0612; A61M 25/0693; A61M 39/24; A61M 2039/0009; A61M 2039/2433; A61M 2205/3331

(57) ABSTRACT

Beedle assemblies with a needle hub with a needle, a catheter tube with a catheter hub and the valve and valve opener positioned in the interior cavity of the catheter hub. The valve can have a valve skirt and a nose section of the valve opener can locate therein. One or more reliefs can be provided with the valve opener so that an elbow or diagonal section on a needle guard can project from a holding space within the valve opener through the relief. The valve opener and the needle guard can be orientated so that the needle guard does not snag the valve opener during retraction.

### FIELD OF ART

The disclosed invention generally relates to needle devices, arterial catheters and intravenous (IV) infusion devices, including IV catheters. In particular, IV catheter assemblies having a valve and a valve actuator for opening the valve are disclosed.

### BACKGROUND

IV catheters are commonly used for a variety of infusion therapies, including infusing fluids into a patient, withdrawing blood from a patient, or monitoring various parameters of the patient's vascular system. Catheters are typically connected to a catheter adapter that accommodates the attachment of IV tubing to the catheter. Blood control catheters include an internal blood control valve that is opened by the insertion of a male Luer or other object into a proximal end of the catheter adapter. Non-limiting examples of blood control valves are disclosed in United States Patent Application Publication No. 2011/0046570, filed Aug. 20, 2009, titled “Systems and Methods for Providing a Flushable Catheter Assembly.” Following placement of the catheter into the vasculature of a patient, an IV fluid source can be connected to the catheter adapter or catheter hub, opening the blood control valve. Thus connected, fluid from the IV source can begin flow into a patient through the catheter.

As is well known in the art, typical blood pressure is 10 to 20 centimeters of water. Infusion bags are usually placed about 100 cm above the patient's heart to direct flow into the patient. At roughly that height, the pressure exerted by the fluid from the infusion bag is much greater than the blood pressure of the patient and therefore can flow into the patient.

Some catheter adapters permit verification of proper placement of the catheter in the blood vessel before fluid infusion begins, such as by providing a flashback chamber of the catheter assembly where a “flashback” of blood can be observed. To confirm flashback in catheter assemblies that do not include a blood control valve, a clinician must manually occlude the vein to prevent undesirable exposure to blood. In contrast, blood control valves can eliminate the need for such manual occlusion, while also reducing the likelihood of blood exposure during catheter placement.

Aspects of the present invention include a catheter assembly comprising a catheter hub, a needle hub, a valve for limiting flow through the catheter hub, a valve opener for opening the valve when advanced by a medical implement, and a needle guard for blocking the needle tip of the needle to prevent inadvertent needle sticks. The valve opener can have plunger elements and the needle guard can have guard arms that are oriented in a manner that avoids snagging between the two during retraction of the needle guard following successful venipuncture.

Aspects of the invention can include a needle assembly comprising: a needle hub with a needle having a needle tip with a needle bevel extending from a distal end of the needle hub; a catheter tube attached to a catheter hub having a body comprising an interior surface defining an interior cavity, the needle extending through the catheter tube and the needle tip out a distal end of the catheter tube in a ready to use position with the needle bevel oriented to a 0-degree position; a valve positioned in the interior cavity of the catheter hub and in contact with an internal shoulder of the catheter hub, said valve comprising an outer perimeter and a wall having three slits defining three flaps; a valve opener sized and shaped to open the three slits to open the valve, said valve opener comprising a nose section having an activation end and a continuous perimeter section having a bore passing therethrough, two plunger elements, including a first plunger element and a second plunger element, each having two lengthwise edges extending proximally of the nose section, and two stabilizer elements; wherein each stabilizer element connects to the two plunger elements so that the two stabilizer elements and the two plunger elements define a stabilizing ring having a continuous surface and the stabilizing ring being spaced from the continuous perimeter section of the nose section by two through passages, which include a first through passage and a second through passage; a needle guard comprising a proximal wall and two resilient arms located in a holding space between the two stabilizer elements, wherein each resilient arm comprises an elbow and the needle guard has two resilient arms and two elbows, which include a first elbow and a second elbow; wherein (1) the first plunger element is oriented to about a 285-degree position to about the 0-degree position and the second plunger element is oriented 180-degrees from the first plunger element, or (2) the first plunger element is oriented to about a 1-degree position to about the 75-degree position and second plunger element is oriented 180-degrees from the first plunger element; and wherein the 0-degree position is a top dead center position of a full circle when looking from an end view of the catheter hub in a distal direction.

Preferably, the first plunger element is oriented to about the 285-degree position to about the 75-degree position including the 0-degree position and second plunger element is oriented 180-degrees from the first plunger element. Preferably, the second plunger element is oriented to about a 105-degree position to about a 255-degree position and the first plunger element is oriented 180-degrees from the first plunger element.

Preferably, the first plunger element is not aligned to the 90-degree position and the second plunger element is not aligned to the 270-degree position. The reference points of the two arms can be a plane drawn through the median of the two arms, in the lengthwise direction. The two points on this plane can represent the two arms, which can be aligned or angled to desired angular positions. The 0-degree position is understood as the top dead center position of a full circle when looking from an end view of the catheter hub in the distal direction.

The first elbow can be located in the first through passage and the second elbow can be located in the second through passage of the valve actuator in the ready to use position.

The two arms of the needle guard can bisect the two plunger elements.

The valve can comprise a proximally facing wall surface and wherein the activation end of the valve opener can be urged against the proximally facing wall surface to push the three flaps against the interior surface of the catheter hub in a used position.

The first elbow can be in contact with a perimeter of the first through passage.

The first elbow can be in contact with the interior surface of the catheter hub.

The first elbow can be in contact with a perimeter of the first through passage and the interior surface of the catheter hub.

The first elbow can be in contact with an edge of the stabilizing ring.

The valve can comprise a valve disc and a valve skirt extending in a proximal direction of the valve disc and defining a valve cavity.

The second elbow can be in contact with the interior surface of the catheter hub.

The nose section of the valve actuator can have a frustoconical shape.

The second elbow can be in contact with a perimeter of the second through passage.

The valve opener can comprise a projection extending radially of a lengthwise axis of the valve opener and the interior surface of the catheter hub can comprise a shoulder for limiting proximal movement of the projection.

The projection on the valve opener or actuator can be a first projection and wherein the valve opener can comprise a second projection spaced from the first projection.

The valve opener can comprise a projection extending radially of a lengthwise axis on an exterior surface of one of the two plunger elements.

A skirt section can extend distally of the wall of the valve.

The first stabilizer element can have a distal edge and the second stabilizer element can have a distal end.

The distal edge of the first stabilizer element can be parallel to the distal edge of the second stabilizing element.

The distal edge of the first stabilizer element can be angled to the distal edge of the second stabilizing element.

A projection can be located on each of the two plunger elements to define two projections, wherein the two projections can be located distally of the stabilizing ring.

The needle guard, the valve, and the valve opener can be entirely located within the interior cavity of the catheter hub.

A protective cap can have a sleeve located around the catheter tube and the needle.

A bushing can wedge a proximal end of the catheter tube against the interior surface of the catheter hub.

A resilient element can be located inside the catheter hub between the bushing and the valve disc, and wherein the resilient element can be sized and shaped to push the valve disc in a proximal direction. For example, the resilient element can push the valve flaps of the valve disc in a proximal direction after the male medical implement has been removed to limit flow across the valve disc.

The resilient element can be an elastic element, such as an O-ring, or a coiled spring.

The wall of the valve can have a generally flat or smooth distally facing wall surface.

The two projections can locate distally of the stabilizing ring.

A method of assembling a needle assembly or manufacturing a needle assembly, which can be a catheter assembly having a needle, the method can comprise: providing a catheter hub with a catheter tube having an open distal end and a proximal end attached to the catheter hub by a bushing, said catheter hub comprising a hub body having an interior surface defining an interior cavity and a proximal opening; positioning a valve comprising an outer perimeter and a wall having three slits defining three flaps in the interior cavity and proximal of the bushing; positioning a valve opener inside the interior cavity of the catheter hub to slidably push open the valve when actuated by a male medical implement, said valve opener comprising a nose section having an activation end and a continuous perimeter section having a bore passing therethrough, two plunger elements, including a first plunger element and a second plunger element, each having two lengthwise edges extending proximally of the nose section, and two stabilizer elements; wherein each stabilizer element connects to the two plunger elements so that the two stabilizer elements and the two plunger elements define a stabilizing ring having a continuous surface and the stabilizing ring being spaced from the continuous perimeter section of the nose section by two through passages, which include a first through passage and a second through passage; placing a needle guard comprising a proximal wall and two resilient arms in a holding space between the two stabilizer elements, wherein each resilient arm comprises an elbow and the needle guard has two resilient arms and two elbows, which include a first elbow and a second elbow; placing a needle, which is attached to a needle hub, through the catheter hub, the valve, and the catheter tube so that a tip of the needle with a needle bevel extends out the open distal end of the catheter tube in a ready to use position; orienting (1) the first plunger element to about a 285-degree position to about the 0-degree position and the second plunger element 180-degrees from the first plunger element, or (2) the first plunger element to about a 1-degree position to about the 75-degree position and second plunger element 180-degrees from the first plunger element; and wherein the 0-degree position is a top dead center position of a full circle when looking from an end view of the catheter hub in a distal direction.

A catheter assembly, which may more broadly be referred to as a needle assembly or a needle device, can comprise a catheter hub with a catheter tube and a bushing. The bushing can be configured to wedge the proximal end of the catheter tube against the interior wall surfaces of the catheter hub to retain the catheter tube to the catheter hub.

Interiorly of the catheter hub, a septum or valve, a valve actuator or valve opener and a safety clip, such as a needle guard or tip protector, can be located. Optionally, the safety clip can be omitted. A needle, which has a change in profile, can be inserted through the proximal opening of the catheter hub with the needle tip protruding from the distal opening of the catheter tube in a ready to use position. A cannula hub or needle hub can attach to the proximal end of the needle and can contact the proximal end of the catheter hub when assembled thereto in the ready to use position. The proximal opening of the catheter hub can be sized with a female Luer taper, optionally with external threads, to engage with a male Luer tip in a Luer slip or a Luer lock. The change in profile can be a crimp or a material buildup.

The tip protector can be configured to cover the needle tip and be removed with the needle following use and the valve and valve actuator remain with the catheter hub for controlling fluid flow therethrough. The valve actuator can be configured to be pushed distally by a male tip into the valve to open the valve for fluid flow, as further discussed below.

A flash back plug or blood stopper assembly can be connected to the needle hub to stop blood flow out the flashback chamber of the needle hub. The flash back plug can be provided at the proximal end the needle hub to allow air to vent but stops blood from spilling out the proximal end of the body of the flash back plug, which can have a chamber and a hydrophobic filter. Alternatively, a syringe can be attached to the proximal end of the needle hub. A second valve and an actuator, described further below, can also be placed within the needle hub.

A protective cap with a sleeve and a saddle can be provided to cover the needle during packaging and before use. The saddle can surround at least part of the catheter hub and the needle hub and be removably engaged to the needle hub. The protective cap can be removed from the catheter assembly prior to use.

In an example in a 360-degree arc or circle, the valve opener can have a pair of plunger elements aligned generally at the 0-degree (or 360-deg.) position and the 180-degree position when the catheter assembly is in the ready to use position and following removal of the needle after successful venipuncture. In other examples, the two plunger elements can be aligned along different angular positions, but not at the 90-degree and 270-degree positions.

The tip protector can have two arms that are generally aligned at a position which is shifted or rotated compared to the first and second plunger elements, preferably by 15° to 90°, in the catheter assembly ready to use position. The tip protector can have two arms that are generally aligned at the 90-degree position and the 270-degree position in the catheter assembly ready to use position. The reference points of the two arms can be a plane drawn through the median of the two arms, in the lengthwise direction. The two points on this plane can represent the two arms, which can be located approximately at the 90-degree position and the 270-degree position. The 0-degree position is understood as the top dead center position of a full circle when looking from an end view of the catheter hub in the distal direction.

The needle bevel at the needle tip can face upwardly, towards the 0-degree position in the ready to use position. These arrangements allow the needle guard or tip protector to be retracted with the needle following successful venipuncture while avoiding or minimizing potential snag between the tip protector and the valve opener in the event the needle guard tilts or pitches as it is being retracted, as further discussed below.

Any number of prior art guards configured for blocking the needle tip of the needle may be used with the present catheter assembly. In the embodiment shown, the tip protector resembles one of the guards shown in U.S. Pat. No. 6,616,630, the contents of which are expressly incorporated herein by reference. In an example, the tip protector can have a proximal wall and two resilient arms and wherein a change in profile on the needle, such as a crimp or a bulge, can engage a perimeter defining an opening on the proximal wall of the tip protector to retract the tip protector in the proximal direction out of the catheter hub following successful venipuncture. The two arms can intersect or they can extend along different sides of the needle and do not intersect along a side view. The tip protector can instead have just one arm.

The two needle guard arms can be spread or biased outwardly by the needle shaft in a ready to use position so that the biased arms of the needle guard engage the inside of the catheter hub, engage the valve opener, such as the edge or edges of the valve opener, or combinations thereof in the ready to use position, as further discussed below. The elbows of the needle guard can alternatively be located within the reliefs of the valve opener but not touch the valve opener or the catheter hub. In an example, only part of the tip protector or needle guard can extend into one or more gaps of the valve opener while the proximal section of the tip protector, such as the proximal wall, can extend proximally or be located proximally of the proximal most surfaces of the tip protector, as further discussed below.

In an example, the needle guard can comprise a proximal wall comprising a perimeter defining an opening. The proximal wall can have a proximally facing wall surface and a distally facing wall surface opposing the proximally facing wall surface. At least one resilient arm can extend distally of the proximal wall. In an example, two resilient arms extend distally of the proximal wall.

One arm can be longer than the other arm on the needle guard. Each arm can include different arm widths, including a first arm section of a first width and a second arm section of a second width, which is smaller than the first width. The two arms can originate from different ends of the proximal wall and can cross one another at their respective second arm sections. The two arms can extend distally of the proximal wall. When viewed from a side along the lengthwise direction of the needle guard, the two arms can intersect one another. When used with a needle, the two arms intersect one another when in a ready to use position and when in the protective position. In an alternative embodiment, the two arms originate from different ends of the proximal wall and extend in a distal direction without crossing one another. The two arms can also have essentially the same arm width along the length of each respective arm.

Two distal walls can be provided, one at an end of each of the two arms. The distal walls can overlap one another along an axial direction of the needle guard by utilizing different arm lengths and/or angling one of the distal walls further distally at an elbow or diagonal section between the distal wall and the resilient arm. In an example, the elbow or diagonal section of each arm, if two arms are utilized, can engage a corresponding guard engagement section inside the catheter hub, such has a groove or a raised projection, inside the valve actuator, or against one or more restraining or restricting surfaces or choke points of one or two reliefs of the valve actuator to removably secure the needle guard inside the catheter hub in the ready position and during the transition process of removing the needle from the catheter hub. The elbows of the needle guard can alternatively be located within the reliefs of the valve opener but not touch the valve opener or the catheter hub.

When the radial profile of the needle guard measured at the one or two elbows reduces in size, such as following movement of the needle tip proximally of the two distal walls, the needle guard can move proximally of the choking or choke point to be removed from the catheter hub with the needle. When the two distal walls move radially, the needle guard reduces its radial profile and can then move through or proximally past the choke point. In forming the needle guard, the needle guard may be folded from a stamped metal sheet to form the guard as shown. Ribs may be formed on the arms, the proximal wall, and/or the distal walls to increase structurally rigidity.

A valve opener can be used with a needle assembly described herein. The valve actuator or opener can have a nose section, a transition section, and two plunger elements extending proximally of the transition section, or proximally from the nose section. The nose section can define a first continuous perimeter section. Other locations of the nose section, away from the first continuous perimeter section, can comprise a slit or a slot.

Two stabilizer elements can attach to the two plunger elements to form a stabilizing ring, which can define a second continuous perimeter section of the valve actuator, in addition to the continuous perimeter section at the nose section. Each stabilizer element can comprise two edges. In an example, the two edges of each stabilizer element can be parallel to one another. The two stabilizer elements can be skewed or slanted so that while two edges of each stabilizer element can be parallel to one another, the two edges from one stabilizer element are not parallel to the two edges of the other stabilizer element. The proximal edges of the two stabilizer elements can be offset along an axial direction or lengthwise direction of the valve actuator. The distal edges of the two stabilizer elements can be offset along an axial direction. In an example, the distal edges can be located obliquely to the axis of the catheter hub.

Two reliefs or two through passages can be provided with the valve opener, each defined or bounded by the transition section, the two plunger elements and the respective stabilizer elements. The two reliefs or through passages may be referred to as a first relief or first through passage and a second relief or second through passage. In an example, each relief or through passage has a perimeter.

In an example, each perimeter of each relief can be defined by the structure of the transition section, the two plunger elements, and the respective stabilizer elements. As the two stabilizer elements can skew or slant in different directions, the two perimeters of the two reliefs or through passages can be different, such as having different perimeter contours or shapes. The edges of the two stabilizer elements are preferably oblique to the direction of the needle or axis of the catheter hub.

The two perimeters of the two reliefs can each be defined by a continuous loop. In other words, the perimeters do not have a slit or a slot to form an open perimeter. However, where a stabilizer element includes a slot or a slit, the perimeter can be an open perimeter or a non-continuous perimeter.

In some examples, the two stabilizer elements can extend laterally without skewing or slanting in the distal direction or the proximal direction. When so configured, the edges of the two stabilizer elements can be parallel to one another. Additionally, the four edges of the two stabilizer elements can be parallel to one another and axially offset. That is, the proximal edge of one stabilizer element can be located more proximally or distally than the proximal edge of the other stabilizer elements while the four edges are parallel to one another.

Two plunger element stubs or extensions can extend proximally of the stabilizing ring. In an example the two plunger element stubs can extend from the stabilizing ring and axially align with the plunger elements. In other examples, the two plunger element stubs are not axially aligned with the two plunger elements. In still other examples, only one plunger element stub aligns with one of the two plunger elements. For a valve opener or actuator with only one plunger element between the first and second continuous perimeter sections, only one of the two plunger element stubs or none of the plunger element stubs can align with the one plunger element.

In some examples, there can be more than two plunger element stubs or extensions extending proximally of the stabilizing ring. The two or more plunger element stubs or extensions can be equally spaced around the proximal periphery of the stabilizing ring or randomly spaced around the proximal periphery of the stabilizing ring. The plunger element stubs can extend the overall length of a valve actuator. The number of plunger element stubs and/or the arc-curve of each plunger element stub, which defines a width of each plunger element stub, can provide a greater overlapping surface with a male Luer tip than fewer numbers or for a plunger element stub with a relatively smaller arc-curve. In other examples, the two plunger element stubs or extension can be considered as part of the two plunger elements and the stabilizer ring is located between two ends of each of the two plunger elements.

The two plunger elements can each have at least two thicknesses to create a projection at an interface between the two thicknesses on an outside surface of each plunger element. The two projections can be located inside a recessed section of the catheter hub so that a shoulder at a proximal end of the recessed section can provide a stop surface to interact with the two projections to prevent dislodgement of the valve opener from the interior of the catheter hub and out from the interior of the catheter hub. In some examples, only one projection is employed on one of the two plunger elements to prevent dislodgement of the valve opener in the proximal direction. In still other examples each plunger element can have a single thickness and the projection is formed by adding material to the plunger element during injection molding at the site of the projection only.

In an example, a planar surface section can be provided with the valve actuator on the same side of each stabilizer element. The planar surface section can originate from about the nose section or the transition section of the valve member and extends proximally to about the two projections.

Two planar surface sections, one on each side of the valve opener corresponding to the two stabilizer elements, can be provided to minimize the overall profile of the transition section and the two plunger elements along a side profile. In an example, the cross-sectional dimension of the nose section, of the transition section, and of the two plunger elements can generally be constant or be the same within typical manufacturing tolerances along a side view, or a 90 degree turn along the lengthwise axis of the valve opener.

A holding space located between two plunger elements, inside the stabilizing ring, between two plunger element stubs, or combinations thereof, can be provided with the valve opener or actuator. Part or all of a needle guard can be located in the holding space in a ready to use position and one or two elbows of the tip protector projecting out the relief. For example, the elbows of the needle guard can be located distal of the two stabilizer elements and located in the through openings or reliefs in the ready to use position. Further, in the ready to use position, the two elbows can contact the interior of the catheter hub, be spaced from the interior of the catheter hub, can contact one or both perimeters of the reliefs, or be spaced from the perimeters of the reliefs and the interior surface of the catheter hub, or combinations thereof. Obviously, if the tip projector only incorporates one arm and one elbow, then the only elbow can similarly be arranged.

The two elbows of the needle guard can be located distal of the two stabilizer elements while the proximal wall of the needle guard can be located proximal of the two stabilizer elements in the ready to use position. In an example, the proximal wall can be flushed with the proximal end surface of a plunger element stub, located proximally of the end surface, or located distally of the end surface. If no plunger element stub is incorporated, a proximal wall of a needle guard can be flushed with the proximal edge of one or both stabilizer elements, located proximally of the proximal edge of one or both stabilizer elements, or located distally of the proximal edge of one or both stabilizer elements.

The distance between the two inside surfaces of the two stabilizer elements can define a choke gap, choke point, or restricting point for a needle guard to limit proximal movement of the needle guard in a ready to use position and/or during retraction of the needle following intravenous access. That is, before the needle tip moves proximally of one or two distal blocking walls of the needle guard, the choke point or gap is too small for the needle guard to pass proximally of the choking point, choke point, or restricting point, due the diagonal measurement between the two elbows being larger than the choke point. However, after the needle tip moves proximally of one or two distal blocking walls of the needle guard and the distal walls are no longer biased by the needle shaft, the two distal walls move radially inwardly to decrease the needle guard's radial profile, which is smaller than the choke point. At that point, with a smaller radial profile measured at the two elbows, the needle guard can move proximally of the choke point to remove the needle guard from the catheter hub along with the needle, such as following successful venipuncture.

A passage can be defined by the wall surface at the nose section of the valve opener. A distal opening opens into the passage from a distal end and the passage has a proximal opening. The needle can be located in or passes through the passage in the ready to use position.

The catheter hub can have an optional pair of wings extending radially of the hub body. When incorporated, the wings can add stability by providing an increased surface area for resting and securing the catheter hub against the skins of a patient, such as following intravenous access.

The plunger elements of the valve opener can be orientated in the 0-deg and 180-deg positions and the bevel at the needle tip can be oriented upwardly and aligning with the upper plunger element. The reference points of the two plunger elements can be a plane drawn through the median of the two plunger elements, in the lengthwise direction. The two points on this plane can represent the two plunger elements, which can be located approximately at the 0-deg and 180-deg positions, or other angular positions as described elsewhere herein. The 0-degree position is understood as the top dead center position of a full circle when looking from an end view of the catheter hub in the distal direction. Further, the two elbows and the two arms of the needle guard can be orientated in the 90-deg and 270-deg positions, and the elbows project out the two reliefs of the valve opener while the bevel at the needle tip can be oriented upwardly and aligned with the push tab on the catheter hub. In an example, the needle bevel can be aligned to the 0-deg position. Still further, the plunger elements of the valve opener can be orientated in the 0-deg and 180-deg positions, the two elbows and the two arms of the needle guard can be orientated in the 90-deg and 270-deg positions, and the elbows can project out the two reliefs of the valve opener while the bevel at the needle tip can be oriented upwardly and aligned with the upper or first plunger element.

In the ready position with the needle hub in contact with the catheter hub and the needle tip extends out the distal end or distal opening of the catheter tube, the catheter assembly is ready for use, such as to perform a venipuncture or intravenous access. Located internally of the catheter hub can be a valve having a valve body with an outer perimeter, a valve disc and a valve skirt extending in an axial direction from the valve disc. In an example, the valve disc can comprise one or more slits defining one or more flaps to be opened by the valve actuator. The type of slits and flaps and the numbers of each incorporated with the valve disc can vary with three slits and three flaps being preferred. In an embodiment, the valve skirt can be positioned in a recessed section formed in the interior cavity of the catheter hub, which can prevent the valve from axially moving once situated inside the catheter hub.

In an example, the recessed section in the interior of the catheter hub has a distal shoulder and a proximal shoulder defining a groove 236 therebetween. The valve skirt has a length that is sized and shaped to fit within the groove or recessed section. In an example, the valve skirt can contact the proximal shoulder while the valve disc can contact the distal shoulder. Viewed differently, the valve located therein can contact the distal shoulder and the proximal shoulder of the recessed section and be restrained thereby in the ready to use position so that the valve skirt is axially fixed or not axially movable.

In an example, the outer diameter or exterior surface of the skirt section can form a snug fit or size-on-size fit with the recessed section of the interior of the catheter hub. In other examples, a slight interference fit can be provided between the two. In still other examples, a small clearance can be provided between the exterior surface of the skirt section and the interior surface at the recessed section with the valve skirt in contact with the proximal shoulder and the valve disc in contact the distal shoulder of the recessed section.

A distal cavity chamber can be provided distal of the valve disc and proximal of the bushing. In some examples, a helical spring or a resilient biasing element, such as an elastomeric ring or cylinder, may be provided in the distal cavity chamber, concentric with the needle, to bias the flaps of the valve disc to assist the valve disc to close the one or more slits.

In an example, the valve disc can comprise a valve diameter, a valve thickness measured orthogonal to the valve diameter, and one or more slits defining two or more flaps, as previously discussed. In the illustrated embodiment, the valve skirt can extend axially to the lengthwise axis of the valve and has an elongated wall that is generally perpendicular to the outer perimeter of the valve disc, forming a generally cylindrical valve body. In some embodiments, the valve skirt may be sloped such that the valve forms a frusto-conical structure.

The valve skirt can define a valve cavity having an open proximal end through which the actuator nose or nose section of the valve actuator can advance and actuate the valve flaps of the valve disc. In one embodiment, at least some part of the actuator nose including the distal actuation end of the valve actuator can be located inside the valve cavity of the valve prior to actuation. Thus, the actuator nose can be narrower than the inside diameter of the valve skirt so that the actuation end fits within the valve cavity and abuts or touches the valve disc in the ready to use position. In an example, the relative dimensions are such that the nose section of the actuator does not touch the interior wall surface of the skirt section with some touching contemplated.

In an example, the actuation end of the valve opener contacts the proximally facing surface of the valve disc in the ready to use position and another part of the valve actuator, such as one or two projections, abuts a shoulder inside the catheter hub to impart a load on the valve disc in a ready to use position but not enough to open the one or more slits, as further discussed below. In other examples, the actuation end can be spaced from the proximally facing surface of the valve disc in the ready to use position and the projection or projections on the actuator either contact the shoulder or are also spaced from the shoulder. When the one or more projections on the actuator are spaced from the shoulder and the actuation end is spaced from the valve disc, the actuator can float within the catheter hub by a small amount in the axial direction.

In an example, a projection extends outwardly from an outer surface of one or both plunger elements. A projection can extend from the outer surface of each plunger element. Each projection resembles a ramp surface having a generally flat edge for abutting the shoulder. The ramp surface of the projection and the direction of the ramp allows the actuator to be inserted into the interior of the catheter hub and be seated within the second recessed section, as further discussed below.

In an example, the actuator or valve opener has a lengthwise axis, the one or more actuating elements can extend axially or parallel to the lengthwise axis. In a particular example, two actuating elements can be diametrically opposed to one another along the lengthwise axis. The two actuating elements can define an outer diameter having a dimension that is larger than the diameter of the nose section.

In an example, the actuating elements are flexible and deflectable so that when pushed by a male Luer tip, the actuating elements defect or flex. The actuating elements can be made deflectable by selecting a material that has the requisite resilient properties. In other examples, the actuating elements can be deflectable by incorporating one or more weakened sections, such as by incorporating a structurally thin section, by incorporating cut-outs, by employing a small cross-section compared to other sections of the same elongated actuating element, or combinations thereof. Alternatively, the actuating elements can be flexible and deflectable by selecting a material that has the requisite resilient properties and by incorporating one or more weakened sections.

In still other examples, each actuating element has more than one different cross-sectional profile or contour along a length section. For example, an elongated plunger element can have a square profile located adjacent a crescent-shaped profile.

The nose section of the valve actuator can be configured to engage the valve to open the valve disc when an axial force is applied by a male tip to the actuating elements towards the distal end of the catheter assembly, such as during the insertion of an IV drip line male Luer connector. Generally, the nose section can be rigid relative to the more pliable valve, which allows the nose section, and more specifically the actuation end, to actuate the valve, such as to deflect the one or more flaps and open the one or more slits on the valve disc. The nose section may be made of a non-compressible material, such as metal, a rigid plastic, or a hard elastomer for pushing against and opening the valve.

In one embodiment, the stabilizers or stabilizer elements can be arc-shaped, forming an arc following the interior profile of the catheter hub and connecting one actuating element to another actuating element. The stabilizers or stabilizer elements may form a substantially cylindrical section on the body of the valve actuator, which body can be spaced apart from the nose section of the valve actuator. In other words, the valve actuator can be elongated and can have sections that are continuous along a radial direction and sections with reliefs or through passages through the wall of the actuator that are not continuous along the radial direction.

In an example, the stabilizers can define a continuous body section or ring along a perimeter or radial direction of the valve actuator that is spaced from a continuous body section of the nose section, which can also be continuous along a perimeter or radial direction. The two stabilizers or stabilizer elements may be joined together with the two plunger elements to form a ring structure. Optionally, the two stabilizers may be slightly offset and angled from each other.

The stabilizers can help the valve actuator remain centered within the catheter hub while the actuator moves, such as when pushed by a male Luer tip. By staying centered, the nose section can be better aligned with the valve disc, such as the slits on the valve disc, allowing for smooth actuation of the valve. The stabilizers can also provide an engagement, via friction, with the interior of the catheter hub to prevent the actuator from sliding in the proximal direction following removal of the male Luer tip.

In one embodiment, the nose section can be configured to remain engaged to the valve disc following actuation of the valve and following removal of the male Luer tip. For example, the nose section can wedge between the one or more slits on the valve disc and be held there by friction, as further discussed below. Surface features, such as bumps, grooves, or barbs, can be provided on the valve actuator, such as on the nose section, to maintain the engagement between the actuator and the valve following actuation and following removal of the male Luer tip.

A relief, opening, or through passage can be provided between the transition section, or from the nose section, and each of the two stabilizers. The two reliefs or through passages can provide clearance so that the interior or central part of the valve actuator and the interior surface of the catheter hub can be in open communication. In other words, between the continuous section of the nose section and the continuous perimeter section defined by the two stabilizers and the plunger elements, call a stabilizing ring or stabilizing ring, are one or two reliefs or openings.

The stabilizing ring of the valve actuator can have an inside diameter that is smaller than the diameter defined by the diagonal section or elbows of the two arms of the needle guard when the two arms are biased outwardly by the side of the needle shaft. Thus, during installation of the needle guard into the holding space of the valve actuator, the diagonal section or elbows of needle guard can deflect to pass through the stabilizing ring and into the open areas defined by the reliefs.

When the tip protector is positioned between the two plunger elements, the two distal walls of the needle guard, more specifically the two diagonal sections or elbows, can be located in the reliefs as discussed above to engage the guard engagement surface on the interior surface of the catheter hub, such as engaging a groove or a projection formed in the interior of the catheter hub, engage the distal edges of the two stabilizer elements, or space from both the stabilizers and the interior surfaces of the catheter hub. The needle guard can therefore be retained within the interior of the catheter hub in the ready to use position and during retraction of the needle following successful venipuncture until the needle tip moves proximal of the two distal walls on the needle guard, at which time the needle guard can close over the needle tip and be removed with the needle.

A second undercut or recessed section proximal of the first recessed section can be provided in the interior cavity of the catheter hub for accommodating the two diagonal sections or elbows. The needle guard can therefore be prevented from sliding in the proximal direction during retraction of the needle following successful venipuncture by a shoulder of the second recessed section or by some other surface feature on the interior of the catheter hub, such as a guard engagement surface on the interior of the catheter hub. Optionally or alternatively, the distal edge of one or both stabilizers can provide the restraining surface to prevent the needle guard from early activation during retraction of the needle, prior to the needle tip moving proximally of the two distal walls. In addition to the distal edge, both stabilizers also have a proximal edge.

In some examples, one or both stabilizer elements can have a slit or a channel, thus dividing the arc-shaped stabilizing element into two. Even with a slit on one or both stabilizer elements, the stabilizing ring, which can be a non-continuous ring, similar to a ring with one or more slots formed through the ring, can still provide the restraining surface to prevent the needle guard from early activation during retraction of the needle, such as prior to the needle tip moving proximally of the two distal walls.

The restraining surface can be referred to as a restrict point, choke gap, or choke point since it provides a rigid structure that prevents the needle guard from moving proximally thereof unless or until the needle guard first activates and collapses radially to reduce its radial profile to then slip proximally of the choke point. In an example, one or two elbows of the needle guard can be restricted by the choke point from moving in the proximal direction until the one or two elbows of the needle guard deflect to reduce needle guard's radial profile. In an example, when the radial profile of the needle guard is reduced, the needle guard can slip through the bore defined by the stabilizing ring, from a distal position of the stabilizing ring to a proximal position of the stabilizing ring.

The valve opener can be made from a metal material or from a plastic material. When made from a metal material, the valve opener can be formed by bending or deep draw methods and the arc shape cross section of the actuating element can provide added rigidity when pushed by the male Luer. Each actuating element can comprise at least two lengthwise edges and a rib can be provided along one or both of the lengthwise edges to further add structural rigidity. One or more gaps can be provided between any two actuating elements. The gaps can provide clearance or space for fluid flow flowing thereacross, such as during flushing blood or IV infusion. The gap between the actuating elements can define a holding space to accommodate a tip protector.

In some embodiments, a majority or most if not all of the tip protector fits within a holding space formed by the body of the actuator, between the two plunger elements, in the ready to use position. This allows the catheter hub to be more compact, as less longitudinal space is needed within the hub to fit both the actuator and the tip protector serially lengthwise or when the two only partially overlap in the axial direction. The tip protector can fit completely within the holding space of the actuator to further reduce the needed space or length in the catheter hub. As shown, the proximal wall of the needle guard can be generally flushed or even with the proximal end surfaces of the two plunger elements.

When the tip protector only engages or contacts the distal edge of the relief or through passage of the actuator, or be spaced form the edges, but not contact the interior surface of the catheter hub, then no deformity or change of diameter is required on the inside wall of the catheter hub and the tip protector can be placed further proximally in the female Luer taper section while complying with the international Luer standard for conical fittings and the overall length of the catheter hub can be further reduced.

The push tab on the catheter hub can be generally located in the 0-deg position relative to the arc circle or full circle end view of the catheter hub. The bevel of the needle tip can be similarly angled and aligned to the 0-degree position, which may also be referred to as a bevel-up position or top dead center position of a circle. In the bevel-up position, the two arms of the needle guard, and similarly the two elbows can be aligned in the 90-deg and 270-deg positions while the two plunger arms can be aligned in the 0-deg and 180-deg positions. This unique arrangement differs from known prior art devices and is incorporated to minimize or avoid having the elbows of the needle guard snag against the stabilizer during retraction of the needle guard following successful venipuncture, where the needle guard can be tilted or pitched by the practitioner during retraction of the needle, as further discussed below.

To align with the needle guard or tip protector, the tip extension at the distal end of the nose section of the needle hub can also be rotated. That is, the tip extension can be oriented to the 90-deg position to push the proximal wall of the needle guard. Thus, while the needle bevel can be oriented in the 0-deg bevel-up position and the tip extension on the needle hub can be oriented in the 90-deg position.

Because of manufacturing tolerances, when a particular angular position is specified, it is understood to mean generally or about and not to be construed as a precise measurement or location unless the context indicates otherwise. While the tip extension at the distal end of the needle hub should be rotated or oriented to align with one of the two arms of the needle guard, the external tab for capturing the catheter hub between the nose section and the external tab can remain at the 180-deg position in the ready to use position or be rotated along with the tip extension.

The tip protector can be substantially fitted within the holding space of the actuator and the proximal wall of the tip protector is approximately even with the proximal ends of the actuating elements, in the axial direction.

During retraction of the needle, the needle shaft can be angled or pitched, intentionally or unintentional such as due to tendency of the practitioner during retraction of the needle following venipuncture. Regardless of why the needle may be angled, the action can cause the needle guard, which can be located over the needle tip, to tilt or pitch, which can cause one or both of the elbows on the two arms to snag the distal edges of the stabilizers of the valve opener if the elbows and the stabilizers are aligned in the 0-degree and 180-degree positions. Said different, if the needle guard is tilted or pitches during retraction, the elbows, if aligned in the 0-deg and 180-deg positions, can snag against the edges of the stabilizers if the valve opener is aligned with the plunger elements in the 90-deg and 270-deg positions.

However, when the elbows are aligned in the 90-deg and 270-deg positions, the two stabilizers can be aligned in the 90-deg and 270-deg positions, and the two plunger elements can be aligned in the 0-deg and 180-deg positions, the tilt or pitch does not cause one or both of the elbows to move against the edges of the stabilizers to snag against the edges of the stabilizers during the retraction. If the elbows were to snag the stabilizers, the needle guard can cause the tip protector to pull against the valve actuator during retraction of the needle to possibly displace the valve actuator from the interior of the catheter hub.

Thus, when the various components are angled, any tilting of the needle during retraction thereof following successful venipuncture, and therefore tilting of the tip protector, will simply cause the tip protector to slide against the inside surface of one or both of the plunger elements. As the inside surfaces of the two plunger elements do not have edges to catch against, the needle guard can readily be retracted away from the valve opener without snagging the valve opener in the process. Thus, the components of the valve opener are configured to operate freely without the elbows snagging the edges of the stabilizers to thereby lead to failure.

After removal of the needle and the needle guard, the valve actuator can be advanced distally into the valve by a syringe tip or a Luer tip of an IV drip line to permit fluid flow through the catheter hub. The Luer tip can be a threaded Luer engaging the catheter hub, which can have a female threaded Luer, in a threaded engagement. Advancing the actuator can cause the actuation end to advance against the valve disc and deflect the flaps radially and distally to open a fluid path to be formed through the catheter assembly. The valve is in a closed position when the one or more slits of the valve disc are closed. In the closed position, the valve can block or limit flow through the catheter hub.

In the valve actuator retracted position, the actuation end of the nose section is not pushed into and opening the one or more slits of the valve. This position is also understood to be the valve pre-activated position or valve closed position. That is, a male Luer tip has not been inserted into the proximal opening of the catheter hub to advance the valve actuator to open the valve. In other words, this is the valve closed position.

When initially inserting the male medical implement, such as the male Luer tip, into the proximal opening of the catheter hub, the male tip initially contacts the two actuating elements on the valve actuator to advance a distally directed force on the two actuating elements to open the valve. The distally directed force moves the actuator in the distal direction until the geometries of the male tip and the proximal opening of the catheter hub stop further distal advancement of the male tip. In an example, a female Luer taper of the catheter hub and a male Luer taper of the male tip mate and block distal advancement of the male tip further into the opening of the catheter hub. A seal is provided by the Luer engagement to prevent fluid from leaking out the proximal opening of the catheter hub.

As the actuator moves distally by the distal advancement of the male tip, such as a syringe tip, the nose of the valve actuator is urged distally and can push against the proximally facing surface of the valve disc. In particular, the nose of the actuator initially pushes against the proximally facing surface of the valve disc and since the valve is axially fixed within the first recessed section of the catheter hub, the one or more flaps on the valve disc can deflect radially and in the distal direction. Fluid from the male tip can then flow through the catheter hub, through the valve, and through the lumen of the catheter tube.

Alternatively, a suction can be applied by the male medical instrument, such as a syringe or vacuum blood collection tube, and blood aspirated from the patient. This is often done for testing samples before infusion therapy is commenced. Also, typically any remaining blood can first be flushed from the inside of the catheter hub before infusion therapy is commenced.

Even after removal of the male tip from the proximal opening of the catheter hub, the valve actuator can remain engaged to the valve. That is, the friction between the valve actuator and the valve disc can exceed restoring forces produced by the valve flaps of the valve in attempting to return to its un-deformed position. This activated position with the engagement can be considered a one-time use or one-time actuation since the valve actuator and valve do not return to the pre-activation position when the male tip is removed.

In an alternative embodiment, the nose section can be configured so that when pushed into the valve during activation, the actuation or activation end does not extend distally past the flaps of the valve disc. This configuration can ensure that the valve actuator is pushed back by the flaps of the valve disc when the male Luer implement is removed. A conical configuration at the distal end of the actuator, i.e., incorporating a tapered nose section, can be such a configuration, which can maintain a proximally directed force vector that is greater than a perpendicular force vector. The angle of the cone can be designed to provide the necessary force vectors when the actuator has reached it maximum distal movement and its minimum distal movement. The difference between the maximum movement and minimum movement of a standard Luer connector is approximately 2.5 millimeters.

Additionally, a helical spring or an elastic element or ring can be incorporated at the distal cavity chamber to increase the re-coil or returning forces of the valve to facilitate pushing the valve actuator in the proximal direction following removal of the male tip to return to the pre-activated position or valve closed position. The elastic element can also help to close the flaps of the valve disc. In this manner, the valve and valve actuator can be re-closed after the initial activation and re-open and so forth, repeatedly. Alternatively or additionally, the flaps on the valve disc can be made thicker to provide enough restoring forces without the need for an elastic element.

A further aspect of a needle guard can include two arms biased apart without the needle that normally biases the arms apart to show features of the distal walls and the curved lips. The arms of the needle guard can have a smooth or flat profile between the elongated arm portion and the elbow with only a single bend or change in direction.

Each elongated arm section of the first arm and the second arm can have sections with different arm widths. Each arm can include a cut-out having a lip that resembles a hook. The cut-out on each arm or cut-outs on the two arms allow the two arms to straddle the needle to provide lateral stability as the needle transitions from the ready to use position, with the needle tip exposed, to the protective position, with the needle tip guarded.

A curved lip on a second arm can be configured to abut the needle at the flat surface adjacent the edge of the second arm when in the ready to use position. The cut-out can be utilized to change the location where the surface of the curved lip, not the edge, contacts the needle. In some examples, the cut-out can be omitted. Ribs can be formed with the two arms, which are optional and can be included to add strengths to the arms, and tabs can extend from edges of the distal walls, which can be optional and can be included to limit sideway pivoting of the needle tip once the needle tip is captured within the needle guard.

In a further aspect of the present embodiment, a catheter hub can have two plunger elements of a valve opener or actuator oriented at other than the 0-deg and 180-deg positions, with 0-degree being at the top dead center of a full arc or circle. For example, the upper or first plunger element can be oriented anywhere from about the 1-degree position to about the 75-degree position and the lower or second plunger element can be oriented 180-degrees from the location of the upper or first plunger element. In an example, the upper plunger element can be oriented at about the 22-degree position and the lower plunger element can be oriented at about the 202-degree position. The reference points of the two plunger elements can be a plane drawn through the median of the two plunger elements, in the lengthwise direction. The two points on this plane can represent the two plunger elements, which can be located approximately at the 22-deg and 202-deg positions.

Thus, when looking at an end view of a catheter assembly in accordance with aspects of the invention, the catheter assembly can comprise a valve actuator having two plunger elements, which can be called a first or upper plunger element and a second or lower plunger element, and wherein the upper plunger element can be oriented from the 0-degree (or 360-deg) position to about the 75-degree position, inclusive, and the lower plunger element can be oriented 180-degrees from the location of the upper plunger element. For example, the first or upper plunger position can be oriented at the 2-deg position, 16-deg position, 38-deg position, 42-deg position, etc. and the lower plunger element can be oriented 180-degrees from the location of the upper plunger element.

Although not shown, the two arms of the needle guard can be oriented between the two plunger arms, approximately bisecting the two plunger arms. The two needle guard arms can be located approximately equally between the two plunger arms.

Thus, when the various components are angled and in accordance with aspects of the invention, any tilting of the needle during retraction thereof following successful venipuncture, and therefore tilting of the tip protector, will simply cause the tip protector to slide against the inside surface of one or both of the plunger elements without snagging. As the inside surfaces 270 of the two plunger elements do not have edges to catch against, the needle guard can readily be retracted away from the valve opener without snagging the valve opener in the process. Thus, when the valve actuator and the needle guard are orientated as shown and described herein, the components of the valve opener are configured to operate freely without the elbows of the needle guard snagging the edges of the stabilizers to thereby lead to failure.

In a further embodiment, the two plunger elements of the valve opener or actuator can be oriented at other than the 0-deg and 180-deg positions. For example, the upper plunger element can be oriented anywhere from about the 285-degree position to about the 359-degree position, inclusive, and the lower plunger element can be oriented 180-degrees from the location of the upper plunger element. For example, the first or upper plunger position can be oriented at the 288-deg position, 297-deg position, 327-deg position, 346-deg position, etc. and the lower plunger element can be oriented 180-degrees from the location of the upper plunger element.

The upper plunger element can be oriented at about the 338-degree position and the lower plunger element is oriented at about the 158-degree position. The reference points of the two plunger elements can be a plane drawn through the median of the two plunger elements, in the lengthwise direction. The two points on this plane can represent the two plunger elements, which can be located approximately at the 338-deg and 158-deg positions.

The two arms of the needle guard can be oriented between the two plunger arms, approximately bisecting the two plunger arms. The two needle guard arms can be located approximately equally between the two plunger arms.

Thus, aspects of the invention include a catheter assembly comprising a valve actuator having two plunger elements, which can be called a first or upper plunger element and a second or lower plunger element, and wherein the upper plunger element can be oriented from the 285-degree position to about the 0-degree position and the lower plunger element can be oriented 180-degrees from the location of the upper plunger element. The 0-degree position is understood as the top dead center position of a full circle when looking from an end view of the catheter hub in the distal direction.

Thus, when the various components are angled and in accordance with aspects of the invention, any tilting of the needle during retraction thereof following successful venipuncture, and therefore tilting of the tip protector, will simply cause the tip protector to slide against the inside surface of one or both of the plunger elements. As the inside surfaces of the two plunger elements do not have edges to catch against, the needle guard can readily be retracted away from the valve opener without snagging the valve opener in the process. Thus, when the valve actuator and the needle guard are orientated as shown and described herein, the components of the valve opener are configured to operate freely without the elbows of the needle guard snagging the edges of the stabilizers to thereby lead to failure.

Methods of making and of using the catheter assemblies and components thereof are within the scope of the present invention.

### BRIEF DESCRIPTION OF THE DRAWINGS

These and other features and advantages of the present devices, systems, and methods will become appreciated as the same becomes better understood with reference to the specification, claims and appended drawings wherein:

FIG. 1 is an exploded perspective view of a catheter assembly provided in accordance with aspects of the invention.

FIG. 2 is a perspective view of a needle guard usable with the catheter assembly of the invention.

FIGS. 3A, 3B, and 3C are different views of a valve opener in accordance with aspects of the invention.

FIG. 4 is a perspective view of a catheter assembly with a removable protective cap.

FIG. 5 is a sectional side perspective view of the catheter assembly of FIG. 4.

FIG. 6 is a detailed partial cross-sectional side view of the catheter assembly of FIG. 4 in a ready to use position, shown without the protective cap.

FIG. 7 is the cross-sectional side view of the catheter assembly of FIG. 6 rotated 90-degrees looking in the distal direction.

FIG. 8 is a detailed partial cross-sectional side perspective view of the catheter assembly of the invention.

FIG. 9 is a cross-sectional side view showing the needle in the process of being removed and the needle guard covering the needle tip.

FIG. 10 shows the needle guard and needle removed and the valve in the closed position.

FIG. 11 shows a male Luer tip inserted into the catheter hub to advance the valve opener to open the valve.

FIGS. 12 and 13 are perspective views of an alternative needle guard in accordance with aspects of the invention.

FIG. 14 is an end view of a catheter assembly shown without a needle or a needle hub.

FIG. 15 is an end view of a catheter assembly shown without a needle or a needle hub.

### DETAILED DESCRIPTION

The detailed description set forth below in connection with the appended drawings is intended as a description of the presently preferred embodiments of catheter assemblies with valves for controlling fluid flow provided in accordance with aspects of the present devices, systems, and methods and is not intended to represent the only forms in which the present devices, systems, and methods may be constructed or utilized. The description sets forth the features and the steps for constructing and using the embodiments of the present devices, systems, and methods in connection with the illustrated embodiments. It is to be understood, however, that the same or equivalent functions and structures may be accomplished by different embodiments that are also intended to be encompassed within the spirit and scope of the present disclosure. As denoted elsewhere herein, like element numbers are intended to indicate like or similar elements or features.

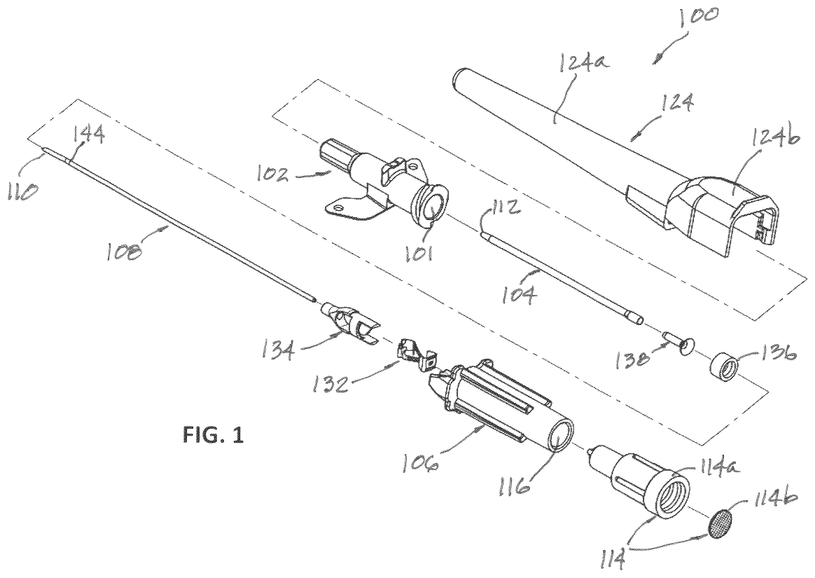

With reference now to FIG. 1, an exploded perspective top view of a catheter assembly 100 provided in accordance with aspects of the present disclosure is shown. As shown, the catheter assembly 100, which may more broadly be referred to as a needle assembly or a needle device, is shown comprising, a catheter hub 102 with a catheter tube 104 and a bushing 138. The bushing 138 can be configured to wedge the proximal end of the catheter tube 104 against the interior wall surfaces of the catheter hub 102 to retain the catheter tube 104 to the catheter hub 102.

Interiorly of the catheter hub 102, a septum or valve 136, an actuator or valve opener 134 and a safety clip 132, such as a needle guard or tip protector, can be located. Optionally, the safety clip 132 can be omitted. A needle 108, which has a change in profile 144, can be inserted through the proximal opening of the catheter hub 102 with the needle tip protruding from the distal opening 112 of the catheter tube in a ready to use position, as shown with reference to FIG. 5-7. A cannula hub or needle hub 106 can attach to the proximal end of the needle 108 and can contact the proximal end of the catheter hub 102 when assembled thereto in the ready to use position. The proximal opening 101 of the catheter hub 102 can be sized with a female Luer taper, optionally with external threads, to engage with a male Luer tip in a Luer slip or a Luer lock.

The tip protector 132 is configured to cover the needle tip and be removed with the needle 108 following use and the valve 136 and valve actuator 134 remain with the catheter hub 102 for controlling fluid flow therethrough. The valve actuator 134 is configured to be pushed distally by a male tip into the valve 136 to open the valve for fluid flow, as further discussed below.

A flash back plug or blood stopper assembly 114 can be connected to the needle hub 106 to stop blood flow out of the flashback chamber 116 of the needle hub 106. The flash back plug 114 can be provided at the proximal end the needle hub 106 to allow air to vent but stops blood from spilling out the proximal end of the body of the flash back plug 114, which has a chamber 114a and a hydrophobic filter 114b. Alternatively, a syringe can be attached to the proximal end of the needle hub 106. A second valve 136 and actuator 134 described further below can also be placed within the needle hub 106.

A protective cap 124 with a sleeve 124a and a saddle 124b can be provided to cover the needle 108 during packaging and before use, which is conventional. The saddle 124b can surround at least part of the catheter hub 102 and the needle hub 106 and be removably engaged to the needle hub. The protective cap 124 is to be removed from the catheter assembly prior to use.

In an example in a 360-degree arc or circle, the valve opener 135 has a pair of plunger elements aligned generally at the 0-degree (or 360-deg.) position and the 180-degree position when the catheter assembly is in the ready to use position and following removal of the needle after successful venipuncture. The tip protector 132 has two arms that are generally aligned at the 90-degree position and the 270-degree position in the catheter assembly ready to use position. The reference points of the two arms can be a plane drawn through the median of the two arms, in the lengthwise direction. The two points on this plane can represent the two arms, which can be located approximately at the 90-degree position and the 270-degree position. The 0-degree position is understood as the top dead center position of a full circle when looking from an end view of the catheter hub in the distal direction. The needle bevel at the needle tip 110 faces upwardly, towards the 0-degree position in the ready to use position. These arrangements allow the needle guard or tip protector 132 to be retracted with the needle following successful venipuncture while avoiding or minimizing potential snag between the tip protector and the valve opener in the event the needle guard tilts or pitches as it is being retracted, as further discussed below.

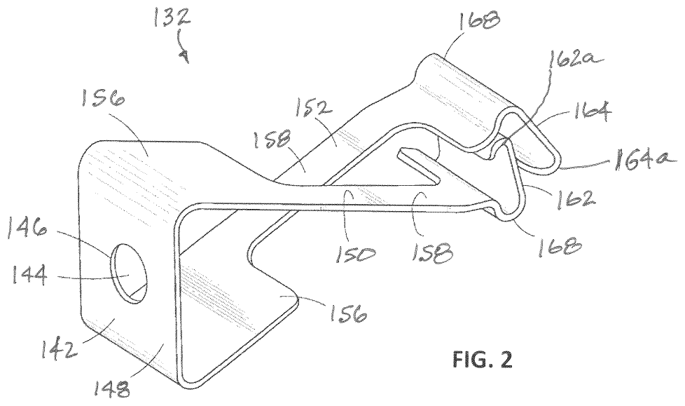

With reference now to FIG. 2, a perspective view of the tip protector 132 of FIG. 1 is shown. Note that while the various features of the tip protector 132 are shown, the tip protector may embody other features not shown or other features that can be modified with the features shown. Further, any number of prior art guards configured for blocking the needle tip 110 of the needle may be used. In embodiment shown, the tip protector 132 resembles one of the guards shown in U.S. Pat. No. 6,616,630, the contents of which are expressly incorporated herein by reference. For example, the tip protector 132 can have a proximal wall and two resilient arms and wherein a change in profile 144 on the needle 108, such as a crimp or a bulge, can engage a perimeter defining an opening on the proximal wall of the tip protector 132 to retract the tip protector in the proximal direction out of the catheter hub following successful venipuncture. The two arms can intersect as described in U.S. Pat. No. 6,616,630 and shown in FIG. 2 or they can extend along different sides of the needle and do not intersect along a side view. The tip protector can instead have just one arm.

The two needle guard arms can be spread or biased outwardly by the needle shaft in a ready to use position so that the biased arms of the needle guard engage the inside of the catheter hub, engage the valve opener, such as the edge or edges of the valve opener, or combinations thereof in the ready to use position, as further discussed below. In an example, only part of the tip protector or needle guard 132 can extend into one or more gaps of the valve opener 134 while the proximal section of the tip protector, such as the proximal wall, can extend proximally or be located proximally of the proximal most surfaces of the tip protector 132, as further discussed below.

FIG. 2 is a rear isometric view of the needle guard 132 of FIG. 1. The needle guard 132 is exemplary only as needle guards with other or different features may be used instead of the exact needle guard 132 shown. In the present embodiment, the needle guard 132 comprises a proximal wall 142 comprising a perimeter 144 defining an opening 146. The proximal wall 142 has a proximally facing wall surface 148 and a distally facing wall surface opposing the proximally facing wall surface. At least one resilient arm 150 extends distally of the proximal wall 142. As shown, two resilient arms 150, 152 extend distally of the proximal wall. One arm can be longer than the other arm. Each arm can also include different arm widths, including a first arm section 156 of a first width and a second arm section 158 of a second width, which is smaller than the first width. The two arms can originate from different ends of the proximal wall 142 and can cross one another at their respective second arm sections 158. Thus, when viewed from a side along the lengthwise direction of the needle guard 132, the two arms intersect one another. When used with a needle, the two arms 150, 152 intersect one another when in a ready to use position and when in the protective position. In an alternative embodiment, the two arms 150, 152 originate from different ends of the proximal wall and extend in a distal direction without crossing one another. Thus, the two arms 150, 152 can also have essentially the same arm width along the length of each respective arm.

Two distal walls 162, 164 are provided, one at an end of each of the two arms 150, 152. The distal walls 162, 164 can overlap one another along an axial direction of the needle guard by utilizing different arm lengths and/or angling one of the distal walls further distally at an elbow or diagonal section 168 between the distal wall and the resilient arm. In an example, the elbow or diagonal section 168 of each arm, if two arms are utilized, can engage a corresponding guard engagement section inside the catheter hub 102, such has a groove or a raised projection, inside the valve actuator 134, or against one or more restraining or restricting surfaces or choke points of one or two reliefs of the valve actuator 134 to removably secure the needle guard inside the catheter hub in the ready position and during the transition process of removing the needle 108 from the catheter hub 102. When the radial profile of the needle guard 132 measured at the one or two elbows 168 reduces in size, such as following movement of the needle tip proximally of the two distal walls 162, 164, the needle guard 132 can move proximally of the choking or choke point to be removed from the catheter hub 102 with the needle. When the two distal walls move radially, the needle guard reduces its radial profile and can then move through or proximally past the choke point. In forming the needle guard, the needle guard 132 may be folded from a stamped metal sheet to form the guard as shown. Ribs may be formed on the arms, the proximal wall, and/or the distal walls to increase structurally rigidity.

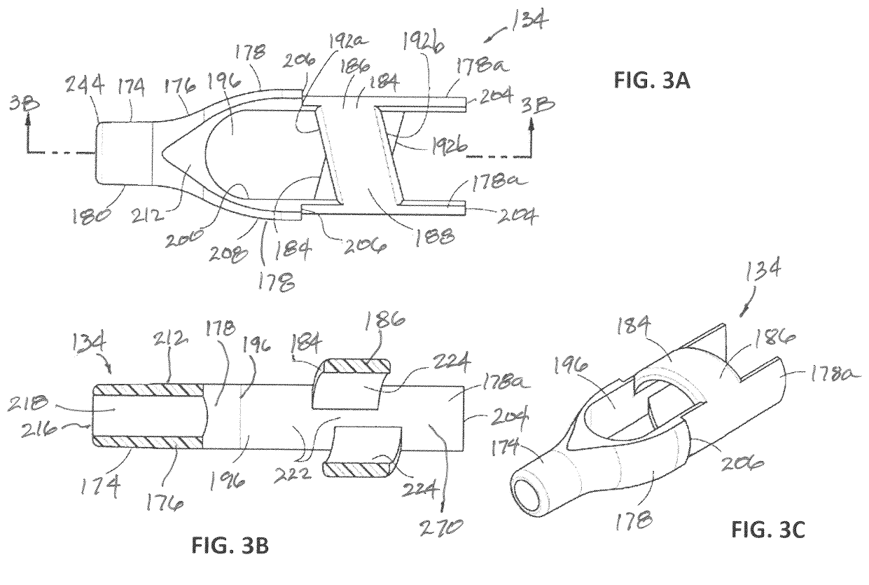

With reference now to FIG. 3A, a top or plan view of a valve opener or actuator 134 is shown. The valve opener can be used with any needle assembly described herein, such as shown in FIG. 5. As shown, the valve actuator has a nose section 174, a transition section 176, and two plunger elements 178, 178 extending proximally of the transition section, or proximally from the nose section. The nose section 174 defines a first continuous perimeter section 180. Other locations of the nose section 174, away from the first continuous perimeter section 180, can comprise a slit or a slot.

Two stabilizer elements 184, 184 are attached to the two plunger elements 178 to form a stabilizing ring 186, which defines a second continuous perimeter section 188 of the valve actuator. Each stabilizer element 184 can comprise two edges 192a, 192b. In an example, the two edges 192a, 192b of each stabilizer element can be parallel to one another. As shown, the two stabilizer elements 184, 184 are skewed or slanted so that while two edges 192a, 192b of each stabilizer element can be parallel to one another, the two edges 192a, 192b from one stabilizer element are not parallel to the two edges 192a, 192b of the other stabilizer element. As shown, the proximal edges 192b of the two stabilizer elements 184, 184 are offset along an axial direction or lengthwise direction of the valve actuator. As shown, the distal edges 192a of the two stabilizer elements 184, 184 are offset along an axial direction. In an example, the distal edges are located obliquely to the axis of the catheter hub.

Two reliefs or two through passages 196 are provided with the valve opener 134, each defined or bounded by the transition section 176, the two plunger elements 178, and the respective stabilizer elements 184, 184. The two reliefs or through passages may be referred to as a first relief or first through passage and a second relief or second through passage. In an example, each relief or through passage has a perimeter 200. In an example, each perimeter can be defined by the structure of the transition section 176, the two plunger elements 178, and the respective stabilizer elements 184, 184. As the two stabilizer elements 184, 184 can skew or slant in different directions, the two perimeters 200 of the two reliefs or through passages 196 can be different, such as having different perimeter contours or shapes. The edges 192a, 192b of the two stabilizer elements 184, 184 are preferably oblique to direction of the needle or axis of the catheter hub. As shown, the two perimeters 200 are each defined by a continuous loop. In other words, in the present embodiment, the perimeters 200 do not have a slit or a slot to form an open perimeter. However, where a stabilizer element 184 includes a slot or a slit, the perimeter can be an open perimeter or a non-continuous perimeter.

In some examples, the two stabilizer elements 184, 184 can extend laterally without skewing or slanting in the distal direction or the proximal direction. When so configured, the edges 192a, 192b of the two stabilizer elements 184, 184 are parallel to one another. Additionally, the four edges of the two stabilizer elements can be parallel to one another and axially offset. That is, the proximal edge 192b of one stabilizer element can be located more proximally or distally than the proximal edge 192b of the other stabilizer element while the four edges are parallel to one another.

With continued reference to FIG. 3A, two plunger element stubs or extensions 178a are shown extending proximally of the stabilizing ring 186. In an example the two plunger element stubs 178a can extend from the stabilizing ring 186 and axially align with the plunger elements 178. In other examples, the two plunger element stubs 178a are not axially aligned with the two plunger elements 178. In still other examples, only one plunger element stub 178a aligns with one of the two plunger elements 178. For a valve opener or actuator with only one plunger element 178 between the first and second continuous perimeter sections, only one of the two plunger element stubs 178a or none of the plunger element stubs can align with the one plunger element.

In some examples, there can be more than two plunger element stubs or extensions 178a extending proximally of the stabilizing ring 186. The two or more plunger element stubs or extensions 178a can be equally spaced around the proximal periphery of the stabilizing ring 186 or randomly spaced around the proximal periphery of the stabilizing ring 186. The plunger element stubs can extend the overall length of a valve actuator. The number of plunger element stubs and/or the arc-curve of each plunger element stub, which defines a width of each plunger element stub, can provide a greater overlapping surface with a male Luer tip than fewer numbers or for a plunger element stub with a relatively smaller arc-curve. In other examples, the two plunger element stubs or extension 178a are considered as part of the two plunger elements 178, 178 and the stabilizer ring 186 is located between two ends of each of the two plunger elements.

With still further reference to FIG. 3A, the two plunger elements 178, 178 are each shown with at least two thicknesses to create a projection 206 at an interface between the two thicknesses on an outside surface 208 of each plunger element. The two projections 206, 206 can be located inside a recessed section of the catheter hub 102 so that a shoulder at a proximal end of the recessed section can provide a stop surface to interact with the two projections 206, 206 to prevent dislodgement of the valve opener 134 from the interior of the catheter hub and out from the interior of the catheter hub. In some examples, only one projection 206 is employed on one of the two plunger elements to prevent dislodgement of the valve opener 134 in the proximal direction. In still other examples each plunger element 178 has a single thickness and the projection 206 is formed by adding material to the plunger element during injection molding at the site of the projection only.

In an example, a planar surface section 212 is provided with the valve actuator 134 on the same side of each stabilizer element 184. The planar surface section 212 originates from about the nose section 174 or the transition section 176 of the valve member and extends proximally to about the two projections 206.

With reference now to FIG. 3B, which is a cross-sectional side view of the valve opener 134 of FIG. 3A taken along lines 3B-3B, the two planar surface sections 212, 212, one on each side of the valve opener 134 corresponding to the two stabilizer elements 184, 184, are provided to minimize the overall profile of the transition section 176 and the two plunger elements 178, 178 along a side profile, as shown in FIG. 3B. Thus, in an example, the cross-sectional dimension of the nose section 174, of the transition section 176, and of the two plunger elements 178, 178 are generally constant or be the same within typical manufacturing tolerances along a side view, or a 90 degree turn along the lengthwise axis of the valve opener from the orientation of FIG. 3A.

With continued reference to FIG. 3B, a holding space 222 is shown, which can be located between two plunger elements 178, inside the stabilizing ring 186, between two plunger element stubs 178a, or combinations thereof. As previously described, part or all of a needle guard 132 can be located in the holding space 222 in a ready to use position and one or two elbows of the tip protector 132 projecting out the relief 196. For example, the elbows of the needle guard can be located distal of the two stabilizer elements 184, 184 and located in the through openings or reliefs 196, 196 in the ready to use position. Further, in the ready to use position, the two elbows can contact the interior of the catheter hub, be spaced from the interior of the catheter hub, can contact one or both perimeters of the reliefs, or be spaced from the perimeters of the reliefs and the interior surface of the catheter hub, or combinations thereof. Obviously, if the tip projector 132 only incorporates one arm and one elbow, then the only elbow can similarly be arranged.

The two elbows 168, 168 of the needle guard can be located distal of the two stabilizer elements 184, 184 while the proximal wall 142 of the needle guard can be located proximal of the two stabilizer elements in the ready to use position. In an example, the proximal wall 142 can be flushed with the proximal end surface 204 of a plunger element stub 178a, located proximally of the end surface 204, or located distally of the end surface. If no plunger element stub is incorporated, a proximal wall of a needle guard 132 can be flushed with the proximal edge of one or both stabilizer elements 184, 184, located proximally of the proximal edge of one or both stabilizer elements 184, 184, or located distally of the proximal edge of one or both stabilizer elements 184, 184.

With still further reference to FIG. 3B, the distance between the two inside surfaces 224, 224 of the two stabilizer elements 184, 184 define a choke gap, choke point, or restricting point for a needle guard 132 to limit proximal movement of the needle guard in a ready to use position and/or during retraction of the needle following intravenous access, as previously discussed. That is, before the needle tip 110 (FIG. 1) moves proximally of one or two distal blocking walls 162, 164 of the needle guard, the choke point or gap is too small for the needle guard to pass proximally of the choking point, choke point, or restricting point, due the diagonal measurement between the two elbows being larger than the choke point. However, after the needle tip moves proximally of one or two distal blocking walls of the needle guard and the distal walls are no longer biased by the needle shaft, the two distal walls move radially inwardly to decrease the needle guard's radial profile, which is smaller than the choke point. At that point, with a smaller radial profile measured at the two elbows, the needle guard can move proximally of the choke point.

A passage 218 is defined by the nose section 174 of the valve opener. A distal opening 216 opens into the passage 218 from a distal end and the passage has a proximal opening. The needle is located in or passes through the passage 218 in the ready to use position.

FIG. 3C is a perspective view of the valve opener 134 of FIGS. 3A and 3B.

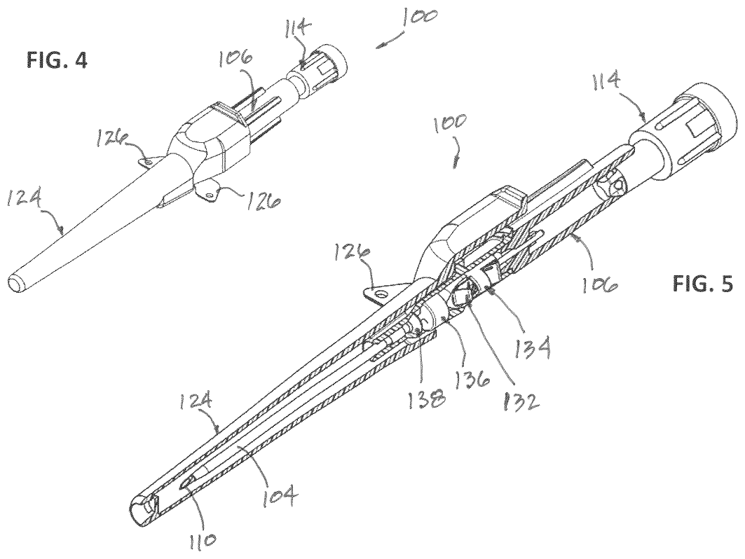

FIG. 4 is an isometric view of the assembled needle assembly 100 of FIG. 1, showing the removable cap 124 disposed over the needle 108 and engaging the needle hub 106. The catheter hub 102 is shown with an optional pair of wings 126, 126 extending radially of the hub body. When incorporated, the wings 126 can add stability by providing an increased surface area for resting and securing the catheter hub 102 against the skins of a patient, such as following intravenous access. FIG. 5 is an isometric cut-away view of the needle assembly 100 of FIGS. 1 and 4. As shown, the plunger elements of the valve opener 134 are orientated in the 0-deg and 180-deg positions and the bevel at the needle tip 110 is oriented upwardly and aligning with the upper plunger element. The reference points of the two plunger elements can be a plane drawn through the median of the two plunger elements, in the lengthwise direction. The two points on this plane can represent the two plunger elements, which can be located approximately at the 0-deg and 180-deg positions. The 0-degree position is understood as the top dead center position of a full circle when looking from an end view of the catheter hub in the distal direction. Further, the two elbows and the two arms of the needle guard 132 are orientated in the 90-deg and 270-deg positions, and the elbows project out the two reliefs 196, 196 of the valve opener 134, while the bevel at the needle tip 110 is oriented upwardly and aligned with the push tab 250 (FIG. 7) on the catheter hub 102. Still further, the plunger elements of the valve opener 134 are orientated in the 0-deg and 180-deg positions, the two elbows and the two arms of the needle guard 132 are orientated in the 90-deg and 270-deg positions, and the elbows project out the two reliefs of the valve opener while the bevel at the needle tip 110 is oriented upwardly and aligning with the upper plunger element.

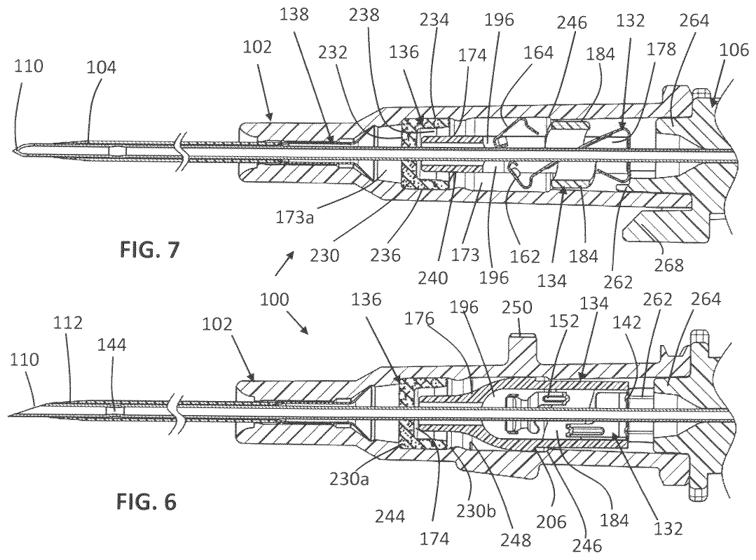

FIG. 6 is a schematic partial cross-sectional side view of the catheter assembly 100 of FIG. 5, shown without the protective cap 124. FIG. 7 is the same cross-sectional side view of the needle assembly 100 of FIG. 6 rotated 90-deg clockwise, looking in the distal direction. The catheter assembly 100 is shown in the two different views with the catheter hub 102 at a proximal end of the catheter tube 104 and the catheter tube extending out a distal end thereof and wherein a needle 108 attached to the needle hub 106 (partially shown) projects through the catheter hub 102 and the catheter tube 104 with the needle tip 110 extending out the distal opening 112 of the catheter tube in the ready to use position.

In the ready position with the needle hub 106 in contact with the catheter hub 102 and the needle tip extending out the distal end or distal opening 112 of the catheter tube 104, the catheter assembly 100 is ready for use, such as to perform a venipuncture or intravenous access. Located internally of the catheter hub 102 is the valve 136 having a valve body 230 with an outer perimeter, a valve disc 232 and a valve skirt 234 extending in an axial direction from the valve disc. In an example, the valve disc 232 comprises one or more slits defining one or more flaps to be opened by the valve actuator 134. The type of slits and flaps and the numbers of each incorporated with the valve disc 232 can vary with three slits and three flaps being preferred. In an embodiment, the valve skirt 234 is positioned in a recessed section 236 formed in the interior cavity 173 of the catheter hub 102, which prevents the valve 136 from axially moving once situated inside the catheter hub 102.

In an example, the recessed section 236 has a distal shoulder 236a and a proximal shoulder 236b (FIG. 6) defining a groove 236 therebetween. The valve skirt 234 has a length that is sized and shaped to fit within the groove or recessed section 236. In an example, the valve skirt 234 contacts the proximal shoulder 236b while the valve disc 232 contacts the distal shoulder 236a. Viewed differently, the valve 136 of FIG. 7 can contact the distal shoulder 236a and the proximal shoulder 236b of the recessed section 236 and be restrained thereby in the ready to use position so that the valve skirt 234 is axially fixed or not axially movable.

In an example, the outer diameter or exterior surface of the skirt section 234 forms a snug fit or size-on-size fit with the recessed section 236 of the interior 130 of the catheter hub 102. In other examples, a slight interference fit is provided between the two. In still other examples, a small clearance is provided between the exterior surface of the skirt section 234 and the interior surface at the recessed section 236 with the valve skirt 234 in contact with the proximal shoulder 236b and the valve disc 232 in contact the distal shoulder 236a of the recessed section.

A distal cavity chamber 173a (FIG. 7) is provided distal of the valve disc 232 and proximal of the bushing 138. In some examples, a helical spring or a resilient biasing element, such as an elastomeric ring or cylinder, may be provided in the distal cavity chamber 173a, concentric with the needle, to bias the flaps of the valve disc 232 to assist the valve disc to close the one or more slits.

In an example, the valve disc 232 comprises a valve diameter, a valve thickness measured orthogonal to the valve diameter, and one or more slits defining two or more flaps, as previously discussed. In the illustrated embodiment, the valve skirt 234 extends axially to the lengthwise axis of the valve and has an elongated wall that is generally perpendicular to the outer perimeter of the valve disc 232, forming a generally cylindrical valve body. In some embodiments, the valve skirt 234 may be sloped such that the valve forms a frusto-conical structure.

The valve skirt 234 defines a valve cavity 238 having an open proximal end 240 through which the actuator nose or nose section 174 of the valve actuator 134 can advance and actuate the valve flaps of the valve disc 232. In one embodiment, at least some part of the actuator nose 174 including the distal actuation end 244 of the valve actuator 134 is located inside the valve cavity 238 of the valve 136 prior to actuation. Thus, the actuator nose 174 can be narrower than the inside diameter of the valve skirt 234 so that the actuation end 244 fits within the valve cavity 238 and abuts or touches the valve disc in the ready to use position. In an example, the relative dimensions are such that the nose section 174 of the actuator does not touch the interior wall surface of the skirt section 234 with some touching contemplated.

In an example, the actuation end 244 of the valve opener contacts the proximally facing surface of the valve disc 232 in the ready to use position and another part of the valve actuator 134, such as one or two projections 206 (FIG. 3A), abuts a shoulder 246 inside the catheter hub to impart a load on the valve disc in a ready to use position but not enough to open the one or more slits, as further discussed below. In other examples, the actuation end 244 is spaced from the proximally facing surface of the valve disc 232 in the ready to use position and the projection or projections 206 on the actuator either contact the shoulder 246 or are also spaced from the shoulder 246. When the one or more projections on the actuator 134 are spaced from the shoulder 246 and the actuation end 244 is spaced from the valve disc 232, the actuator can float within the catheter hub by a small amount in the axial direction.

In an example, a projection 206 extends outwardly from an outer surface of one or both plunger elements 178. As shown, a projection 206 extends from the outer surface of each plunger element 178. Each projection 206 resembles a ramp surface having a generally flat edge for abutting the shoulder 246. The ramp surface of the projection 206 and the direction of the ramp allows the actuator 134 to be inserted into the interior of the catheter hub and be seated within the second recessed section 240, as further discussed below.

In an example, the actuator or valve opener 134 has a lengthwise axis, the one or more actuating elements 178 extend axially or parallel to the lengthwise axis. In a particular example, two actuating elements 178 are diametrically opposed to one another along the lengthwise axis. As shown, the two actuating elements 178 define an outer diameter having a dimension that is larger than the diameter of the nose section 174.

In an example, the actuating elements 178 are flexible and deflectable so that when pushed by a male Luer tip, the actuating elements defect or flex. The actuating elements are deflectable by selecting a material that has the requisite resilient properties. In other examples, the actuating elements are deflectable by incorporating one or more weakened sections, such as by incorporating a structurally thin section, by incorporating cut-outs, by employing a small cross-section compared to other sections of the same elongated actuating element, or combinations thereof. Alternatively, the actuating elements 178 can be flexible and deflectable by selecting a material that has the requisite resilient properties and by incorporating one or more weakened sections.

In still other examples, each actuating element 178 has more than one different cross-sectional profile or contour along a length section. For example, an elongated plunger element can have a square profile located adjacent to a crescent-shaped profile.

The nose section 174 of the valve actuator 134 can be configured to engage the valve 136 to open the valve disc 232 when an axial force is applied by a male tip to the actuating elements 178 towards the distal end of the catheter assembly 100, such as during the insertion of an IV drip line male Luer connector. Generally, the nose section 174 is rigid relative to the more pliable valve 136, which allows the nose section 174, and more specifically the actuation end 244, to actuate the valve 136, such as to deflect the one or more flaps and open the one or more slits on the valve disc 232. The nose section 174 may be made of a non-compressible material, such as metal, a rigid plastic, or a hard elastomer for pushing against and opening the valve.

In one embodiment, the stabilizers or stabilizer elements 184, 184 are arc-shaped, forming an arc following the interior profile of the catheter hub 102 and connecting one actuating element 178 to another actuating element 178. The stabilizers or stabilizer elements 184 may form a substantially cylindrical section on the body of the valve actuator, which body is spaced apart from the nose section 174 of the valve actuator. In other words, the valve actuator can be elongated and can have sections that are continuous along a radial direction and sections with reliefs or through passages through the wall of the actuator that are not continuous along the radial direction.

In an example, the stabilizers 184, 184 define a continuous body section or ring 186 along a perimeter or radial direction of the valve actuator that is spaced from a continuous body section of the nose section 174, which is also continuous along a perimeter or radial direction. The two stabilizers or stabilizer elements 184, 184 may be joined together with the two plunger elements 178 to form a ring structure. Optionally, the two stabilizers may be slightly offset and angled from each other.

The stabilizers 184 can help the valve actuator 134 remain centered within the catheter hub 102 while the actuator moves, such as when pushed by a male Luer tip. By staying centered, the nose section 174 can be better aligned with the valve disc 232, such as the slits on the valve disc, allowing for smooth actuation of the valve 136. The stabilizers 184 can also provide an engagement, via friction, with the interior of the catheter hub 102 to prevent the actuator 134 from sliding in the proximal direction following removal of the male Luer tip.

In one embodiment, the nose section 174 is configured to remain engaged to the valve disc 232 following actuation of the valve and following removal of the male Luer tip. For example, the nose section 174 can wedge between the one or more slits on the valve disc and be held there by friction, as further discussed below. Surface features, such as bumps, grooves, or barbs, can be provided on the valve actuator 134, such as on the nose section, to maintain the engagement between the actuator and the valve following actuation and following removal of the male Luer tip.

A relief, opening, or through passage 196 can be provided between the transition section 176, or from the nose section, and each of the two stabilizers 184, 184. The two reliefs or through passages 196 can provide clearance so that the interior or central part of the valve actuator 134 and the interior surface of the catheter hub 102 can be in open communication. In other words, between the continuous section of the nose section and the continuous perimeter section defined by the two stabilizers 184, 184 and the plunger elements 178, call a stabilizing ring or stabilizing ring 186, are one or two reliefs or openings 196.

The stabilizing ring 186 of the valve actuator 134 can have an inside diameter that is smaller than the diameter defined by the diagonal section or elbows 168 of the two arms 150, 178 of the needle guard 132 when the two arms are biased outwardly by the side of the needle shaft. Thus, during installation of the needle guard 132 into the holding space of the valve actuator 134, the diagonal section or elbows of needle guard 132 can deflect to pass through the stabilizing ring 186 and into the open areas defined by the reliefs 196.

When the tip protector 132 is positioned between the two plunger elements 178, the two distal walls 162, 164 of the needle guard 132, more specifically the two diagonal sections or elbows 168, can be located in the reliefs 196 as discussed above to engage the guard engagement surface on the interior surface of the catheter hub 102, such as engaging a groove or a projection formed in the interior of the catheter hub, engage the distal edges of the two stabilizer elements, or space from both the stabilizers and the interior surfaces of the catheter hub. The needle guard can therefore be retained within the interior of the catheter hub in the ready to use position and during retraction of the needle following successful venipuncture until the needle tip moves proximal of the two distal walls on the needle guard, at which time the needle guard can close over the needle tip and be removed with the needle as discussed above with reference to FIG. 3.

A second undercut or recessed section 248 proximal of the first recessed section 236 can be provided in the interior cavity 173 of the catheter hub 102 for accommodating the two diagonal sections or elbows 168. The needle guard 132 can therefore be prevented from sliding in the proximal direction during retraction of the needle following successful venipuncture by a shoulder 246 of the second recessed section 248 or by some other surface feature on the interior of the catheter hub, such as a guard engagement surface on the interior of the catheter hub. Optionally or alternatively, the distal edge 192a of one or both stabilizers 184, 184 can provide the restraining surface to prevent the needle guard 132 from early activation during retraction of the needle, prior to the needle tip moving proximally of the two distal walls 162, 164. In addition to the distal edge 192a, both stabilizers 184, 184 also have a proximal edge 192b.

In some examples, one or both stabilizer elements 184, 184 can have a slit or a channel, thus dividing the arc-shaped stabilizing element into two. Even with a slit on one or both stabilizer elements 184, 184, the stabilizing ring 186, which can be a non-continuous ring, similar to a ring with one or more slots formed through the ring, can still provide the restraining surface to prevent the needle guard 132 from early activation during retraction of the needle, such as prior to the needle tip moving proximally of the two distal walls 162, 164. The restraining surface can also be referred to as a restrict point, choke gap, or choke point since it provides a rigid structure that prevents the needle guard from moving proximally thereof unless or until the needle guard first activates and collapses radially to reduce its radial profile to then slip proximally of the choke point. In an example, one or two elbows 168 of the needle guard can be restricted by the choke point from moving in the proximal direction until the one or two elbows of the needle guard deflect to reduce needle guard's radial profile. In an example, when the radial profile of the needle guard is reduced, the needle guard can slip through the bore defined by the stabilizing ring, from a distal position of the stabilizing ring to a proximal position of the stabilizing ring.

The valve opener 134 can be made from a metal material or from a plastic material. When made from a metal material, the valve opener 134 can be formed by bending or deep draw methods and the arc shape cross section of the actuating element 178 can provide added rigidity when pushed by the male Luer. Each actuating element 178 can comprise at least two lengthwise edges and a rib can be provided along one or both of the lengthwise edges to further add structural rigidity. One or more gaps can be provided between any two actuating elements 178. The gaps can provide clearance or space for fluid flow flowing thereacross, such as during flushing blood or IV infusion. The gap between the actuating elements 178 can define a holding space 222 to accommodate a tip protector 132.

In some embodiments, a majority or most if not all of the tip protector 132 fits within a holding space 222 formed by the body of the actuator 134, between the two plunger elements 178, in the ready to use position, as shown in FIG. 6. This allows the catheter hub 102 to be more compact, as less longitudinal space is needed within the hub to fit both the actuator 134 and the tip protector 132 serially lengthwise or when the two only partially overlap in the axial direction. In FIG. 6, the tip protector 132 is shown fitting completely within the holding space of the actuator 134 to further reduce the needed space or length in the catheter hub. As shown, the proximal wall 142 of the needle guard 132 is generally flushed or even with the proximal end surfaces of the two plunger elements 178.

When the tip protector 132 only engages or contacts the distal edge 192a of the relief or through passage 196 of the actuator, or be spaced form the edges, but not contact the interior surface of the catheter hub, then no deformity or change of diameter is required on the inside wall of the catheter hub and the tip protector 132 can be placed further proximally in the female Luer taper section while complying with the international Luer standard for conical fittings and the overall length of the catheter hub 102 can be further reduced.

As shown in the FIG. 6 view, the push tab 250 on the catheter hub 102 is generally located in the 0-deg position relative to the arc circle end view of the catheter hub 102. The bevel of the needle tip 110 is similarly angled and aligned to the O-degree position, which may also be referred to as a bevel-up position. In the bevel-up position, the two arms 150, 152 of the needle guard, and similarly the two elbow 168, 168, are aligned in the 90-deg and 270-deg positions while the two plunger arms 178, 178 are aligned in the 0-deg and 180-deg positions. This unique arrangement differs from known prior art devices and is incorporated to minimize or avoid having the elbows snag against the stabilizer during retraction of the needle guard following successful venipuncture, where the needle guard 132 can be tilted or pitched by the practitioner, as further discussed below.

To align with the needle guard or tip protector 132, the tip extension 262 at the distal end of the nose section 264 of the needle hub 106 is also rotated. That is, the tip extension 262 is oriented to the 90-deg position to push the proximal wall of the needle guard. Thus, while the needle bevel is oriented in the 0-deg bevel-up position, the tip extension 262 on the needle hub is oriented in the 90-deg position. Because of manufacturing tolerances, when a particular angular position is specified, it is understood to mean generally or about and not to be construed as a precise measurement or location unless the context indicates otherwise. While the tip extension 262 at the distal end of the needle hub 106 should be rotated or oriented to align with one of the two arms 150, 152 of the needle guard 132, the external tab 268 for capturing the catheter hub between the nose section 264 and the external tab 268 can remain at the 180-deg position in the ready to use position or be rotated along with the tip extension 262.

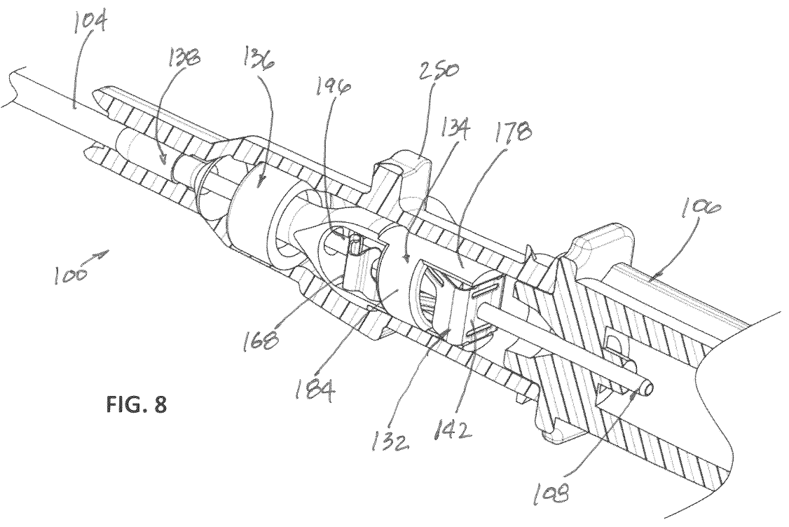

With reference now to FIG. 8, a perspective view of the catheter assembly 100 of FIGS. 6 and 7 is shown without the tip extension 262. One of the reliefs or through passages 196 is clearly shown proximal of the nose section 174 of the actuator 134 and having an elbow 168 of one of the arms located therein to engage with the interior of the catheter hub 102, the distal edge of the stabilizer 184, neither the interior surface or the distal edge, or combinations thereof, as previously discussed. In the illustrated embodiment, the tip protector 132 substantially fits within the holding space of the actuator 134 and the proximal wall 142 of the tip protector is approximately even with the proximal ends of the actuating elements 178, in the axial direction.

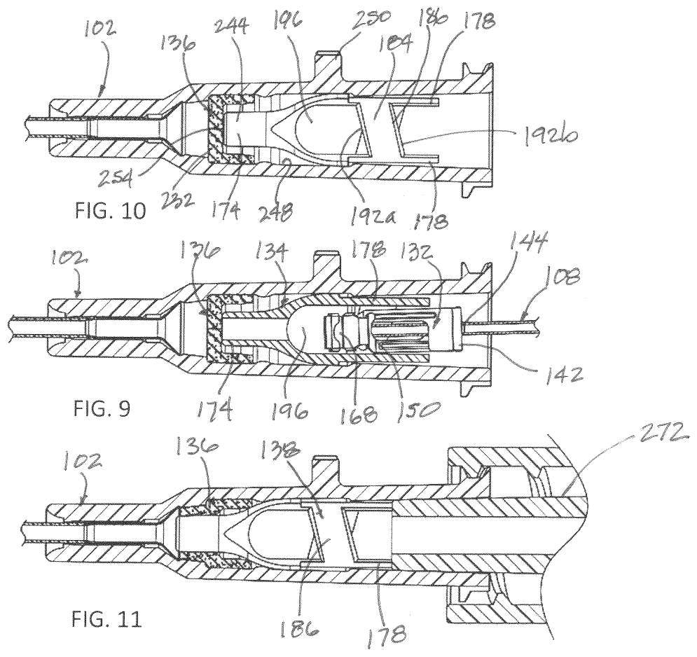

With reference now to FIG. 9 in combination with FIGS. 5-8, a schematic view showing the needle 108 retracted proximally at an angle above the center line following successful venipuncture is shown. The figure shows the needle tip 110 (FIG. 1) moving proximally of the two distal walls of the needle guard 132 and the needle guard blocking the needle tip. The angle of the needle shaft can be positioned intentionally or unintentional, such as due to tendency of the practitioner during retraction of the needle following venipuncture. Regardless of why the needle may be angled, the action causes the needle guard 132, which is located over the needle tip, to tilt or pitch, which can cause one or both of the elbows 168, 168 on the two arms to snag the distal edges 192 a, 192 a of the stabilizers 184, 184 if the elbows and the stabilizers are aligned in the 0-degree and 180-degree positions. Said different, if the needle guard 132 is tilted or pitches during retraction, the elbows, if aligned in the 0-deg and 180-deg positions, can snag against the edges of the stabilizers if the valve opener is aligned with the plunger elements in the 90-deg and 270-deg positions. Note that FIG. 9 is shown without one of the stabilizer elements 186 for clarity.

However, when the elbows 168, 168 are aligned in the 90-deg and 270-deg positions, the two stabilizers 184, 184 are aligned in the 90-deg and 270-deg positions, and the two plunger elements 178, 178 are aligned in the 0-deg and 180-deg positions, as shown in FIG. 8, the tilt or pitch does not cause one or both of the elbows to move against the edges of the stabilizers to snag against the edges of the stabilizers during the retraction. If the elbows were to snag the stabilizers, the needle guard can cause the tip protector 132 to pull against the valve actuator 134 during retraction of the needle to possibly displace the valve actuator 134 from the interior of the catheter hub. Thus, when the various components are angled as shown in FIG. 8 and in accordance with aspects of the invention, any tilting of the needle 108 during retraction thereof following successful venipuncture, and therefore tilting of the tip protector 132, will simply cause the tip protector to slide against the inside surface 270 (FIG. 3B) of one or both of the plunger elements 178, 178. As the inside surfaces 270 of the two plunger elements 178, 178 do not have edges to catch against, the needle guard 132 can readily be retracted away from the valve opener without snagging the valve opener in the process. Thus, when orientated as shown in FIG. 8 and elsewhere, the components of the valve opener are configured to operate freely without the elbows snagging the edges of the stabilizers to thereby lead to failure.

FIG. 10 is a partial cross-sectional side view of the catheter assembly 100 following removal of the needle 108 and the needle guard 132, and with both stabilizer elements 186 shown. FIG. 11 shows the valve actuator 134 advanced distally into the valve 136 by a syringe tip or a Luer tip 272 of an IV drip line to permit fluid flow through the catheter hub. The Luer tip 272 can be a threaded Luer engaging the catheter hub 102, which can have a female threaded Luer, in a threaded engagement. Advancing the actuator 134 causes the actuation end 244 to advance against the valve disc 232 and deflecting the flaps radially and distally to open a fluid path to be formed through the catheter assembly 100. As shown, the valve 136 is in a closed position in which the one or more slits of the valve disc 232 are closed. Also shown is the valve actuator 134 retracted position in which the actuation end 244 of the nose section 174 is not pushed into and opening the one or more slits. This position is also understood to be the valve pre-activated position. That is, a male Luer tip has not been inserted into the proximal opening of the catheter hub to advance the valve actuator to open the valve. In other words, this is the valve closed position.

When initially inserting the male medical implement 272, such as the male Luer tip, into the proximal opening of the catheter hub 102, the male tip initially contacts the two actuating elements 178 on the valve actuator 134 to advance a distally directed force on the two actuating elements 178 to open the valve 136. The distally directed force moves the actuator 134 in the distal direction until the geometries of the male tip and the proximal opening of the catheter hub stop further distal advancement of the male tip. In an example, a female Luer taper of the catheter hub 102 and a male Luer taper of the male tip mate and block distal advancement of the male tip further into the opening of the catheter hub. A seal is provided by the Luer engagement to prevent fluid from leaking out the proximal opening of the catheter hub.

As the actuator 134 moves distally by the distal advancement of the male tip, the nose 174 of the valve actuator 134 is urged distally and pushes against the proximally facing surface of the valve disc 232. In particular, the nose of the actuator 134 initially pushes against the proximally facing surface of the valve disc 232 and since the valve is axially fixed within the first recessed section 236 of the catheter hub, the one or more flaps on the valve disc 232 deflect radially and in the distal direction. Fluid from the male tip can then flow through the catheter hub 102, through the valve 136, and through the lumen of the catheter tube 104.

Alternatively, a suction can be applied by the male medical instrument, such as a syringe or vacuum blood collection tube, and blood aspirated from the patient. This is often done for testing samples before infusion therapy is commenced. Also, typically any remaining blood is first flushed from the inside of the catheter hub 102 before infusion therapy is commenced.

FIG. 11 shows the actuation end 244 of the nose section 174 projected through the valve disc 232 and either contacts or is just about to contact the bushing 138 in the valve actuated position. In this configuration, even after removal of the male tip, the valve actuator 134 can remain engaged to the valve 136. That is, the friction between the valve actuator 134 and the valve disc 232 exceeds the restoring forces produced by the valve flaps of the valve in attempting to return to its un-deformed position shown in FIG. 10. This activated position can be considered a one-time use or one-time actuation since the valve actuator 134 and valve 136 do not return to the pre-activation position of FIG. 10 when the male tip is removed.

In an alternative embodiment, the nose section 174 is configured so that when pushed into the valve 136 during activation, the actuation or activation end 244 does not extend distally past the flaps of the valve disc 232. This configuration can ensure that the valve actuator 134 is pushed back by the flaps of the valve disc when the male Luer implement is removed. A conical configuration at the distal end of the actuator 134, i.e., incorporating a tapered nose section 174, can be such a configuration, which can maintain a proximally directed force vector that is greater than a perpendicular force vector. The angle of the cone can be designed to provide the necessary force vectors when the actuator 134 has reached its maximum distal movement and its minimum distal movement. The difference between the maximum movement and minimum movement of a standard Luer connector is approximately 2.5 millimeters.

Additionally, a spring or an elastic element or ring can be incorporated at the distal cavity chamber 173a to increase the re-coil or returning forces of the valve 136 to facilitate pushing the valve actuator 134 in the proximal direction following removal of the male tip to return to the pre-activated position or valve closed position shown in FIG. 10. The elastic element can also help to close the flaps of the valve disc 232. In this manner, the valve 136 and valve actuator 134 can be re-closed after the initial activation and re-open and so forth, repeatedly. Alternatively, or additionally, the flaps on the valve disc can be made thicker to provide enough restoring forces without the need for an elastic element.

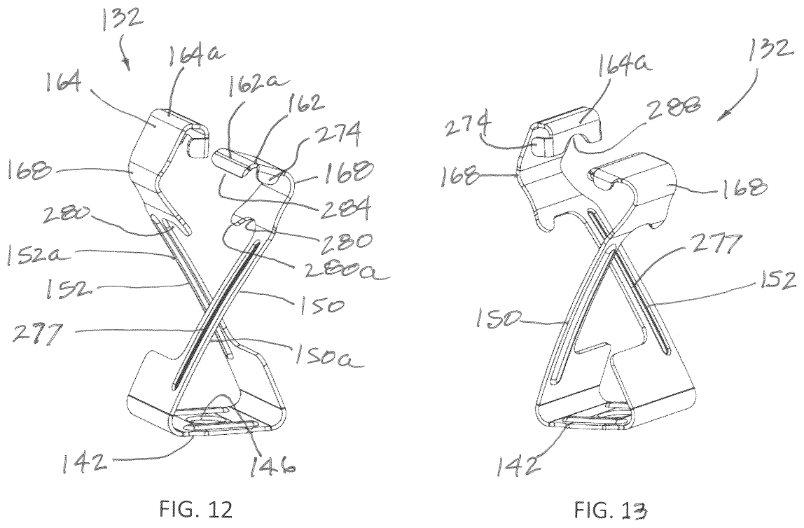

With reference now to FIGS. 12 and 13, two different perspective views of a needle guard 132 provided in accordance with alternative aspects of the invention are shown. The needle guard 132 of the present embodiment is similar to the needle guard shown with respect to FIG. 2 and elsewhere and can be used with various catheter assemblies described herein. The two arms 150, 152 are shown biased apart without the needle that normally biases the arms apart to show features of the distal walls 164, 164 and the curved lips 164a, as further discussed below. The arms 150, 152 of the needle guard 132 are shown with a smooth or flat profile between the elongated arm portion 150a, 152a and the elbow 168, 168, with only a single bend or change in direction. As shown, each elongated arm section 150a, 152a of the first arm 150 and the second arm 152 has sections with different arm widths. Each arm 150, 152 also includes a cut-out 280 having a lip 280a that resembles a hook. The cut-out or cut-outs 280, 280 on the two arms allow the two arms 150, 152 to straddle the needle to provide lateral stability as the needle transitions from the ready to use position, with the needle tip exposed, to the protective position, with the needle tip guarded.

FIG. 12 shows the curved lip 162a on the first arm 150 with a straight edge 284. However, the curved lip 162a is configured to abut the needle at the flat surface adjacent the edge when in the ready to use position. FIG. 13 shows the curved lip 164a on the second arm 152 with a cut-out 288 resembling an arc. The cut-out 288 can be utilized to change the location where the surface of the curved lip 162a, not the edge, contacts the needle. In some examples, the cut-out 288 can be omitted. Also shown are ribs 277 formed with the two arms, which are optional and can be included to add strengths to the arms, and tabs 274 extending from edges of the distal walls 164, 164, which are also optional and can be included to limit sideway pivoting of the needle tip once the needle tip is captured within the needle guard.

The needle guard 132 of FIGS. 12 and 13, as well as other needle guards described herein, can each be made from a stamped metal sheet, such as a stamped stainless-steel sheet, and using a stamping and bending method to form the needle guard in the shape shown.

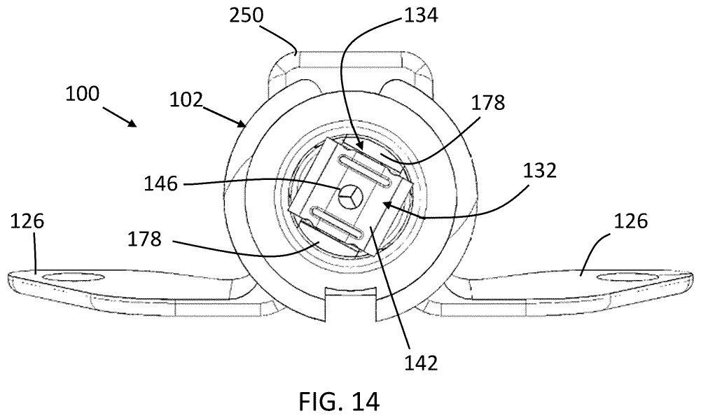

With reference now to FIG. 14, a needle hub 102 is shown without a needle and a needle hub to show the needle guard 132 and the two plunger elements 178, 178 of the actuator or valve opener 134 in accordance with further aspects of the invention. The present catheter assembly 100 is similar to the catheter assembly of FIG. 4-11, with a few exceptions. In the present embodiment, the two plunger elements 178, 178 of the valve opener or actuator 134 can be oriented at other than the 0-deg and 180-deg positions, with 0-degree being at the top dead center of a full arc or circle. To make expressed, the angular position is the positions of a mid-point of a width of the first plunger element and a mid-point of a width the second plunger element the circular radiant relative to the degrees of the circle. The degree can be specified from the perspective when looking in the distal direction or the proximal direction. For example, the upper plunger element 178 can be oriented anywhere from about the 1-degree position to about the 75-degree position, inclusive, and the lower plunger element can be oriented 180-degrees from the location of the upper plunger element 178, when looking in the distal direction of FIG. 14. As shown, the upper plunger element 178 is oriented at about the 22-degree position and the lower plunger element 178 is oriented at about the 202-degree position. The reference points of the two plunger elements 178, 178 can be a plane drawn through the median of the two plunger elements, in the lengthwise direction. The two points on this plane can represent the two plunger elements, which can be located approximately at the 22-deg and 202-deg positions, when looking in the distal direction.

Thus, when looking at the embodiments of FIG. 11 and FIG. 14, aspects of the invention include a catheter assembly comprising a valve actuator having two plunger elements 178, 178, which can be called a first or upper plunger element and a second or lower plunger element, and wherein the upper plunger element 178 can be oriented from about the 0-degree (or 360-deg) position to about the 75-degree position, inclusive, and the lower plunger element 178 can be oriented 180-degrees from the location of the upper plunger element 178. For example, the first or upper plunger position can be oriented at the 2-deg position, 16-deg position, 38-deg position, 42-deg position, etc. and the lower plunger element can be oriented 180-degrees from the location of the upper plunger element.

Although not shown, the two arms of the needle guard 132 can be oriented between the two plunger arms 178, 178, approximately bisecting the two plunger arms. The two needle guard arms can be located approximately equally between the two plunger arms.

Thus, when the various components are angled as shown in FIG. 14 and in accordance with aspects of the invention, any tilting of the needle 108 during retraction thereof following successful venipuncture, and therefore tilting of the tip protector 132, will simply cause the tip protector to slide against the inside surface 270 (FIG. 3B) of one or both of the plunger elements 178, 178. As the inside surfaces 270 of the two plunger elements 178, 178 do not have edges to catch against, the needle guard 132 can readily be retracted away from the valve opener without snagging the valve opener in the process. Thus, when the valve actuator and the needle guard are orientated as shown in FIG. 14, the components of the valve opener are configured to operate freely without the elbows of the needle guard snagging the edges of the stabilizers to thereby lead to failure.

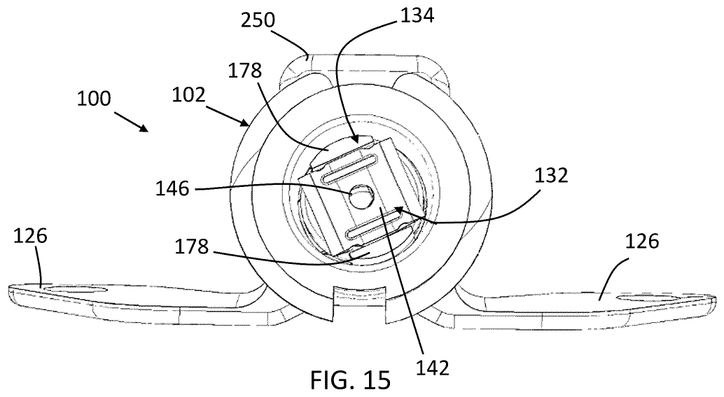

With reference now to FIG. 15, a needle hub 102 is shown without a needle and a needle hub to show the needle guard 132 and the two plunger elements 178, 178 of the actuator or valve opener 134 in accordance with further aspects of the invention. The present catheter assembly 100 is similar to the catheter assembly of FIG. 4-11 and FIG. 14, with a few exceptions. In the present embodiment, the two plunger elements 178, 178 of the valve opener or actuator 134 can be oriented at other than the 0-deg and 180-deg positions. For example and as discussed elsewhere regarding relative degree positions, the upper plunger element 178 can be oriented anywhere from about the 285-degree position to about the 359-degree position, inclusive, and the lower plunger element can be oriented 180-degrees from the location of the upper plunger element 178. For example, the first or upper plunger position can be oriented at the 288-deg position, 294-deg position, 307-deg position, 326-deg position, etc. and the lower plunger element can be oriented 180-degrees from the location of the upper plunger element. As shown, the upper plunger element 178 is oriented at about the 338-degree position and the lower plunger element 178 is oriented at about the 158-degree position. The reference points of the two plunger elements 178, 178 can be a plane drawn through the median of the two plunger elements, in the lengthwise direction. The two points on this plane can represent the two plunger elements, which can be located approximately at the 338-deg and 158-deg positions.

Although not shown, the two arms of the needle guard 132 can be oriented between the two plunger arms 178, 178, approximately bisecting the two plunger arms. The two needle guard arms can be located approximately equally between the two plunger arms.

Thus, when looking at the embodiments of FIG. 11 and FIG. 15, aspects of the invention include a catheter assembly comprising a valve actuator having two plunger elements 178, 178, which can be called a first or upper plunger element and a second or lower plunger element, and wherein the upper plunger element 178 can be oriented from about the 285-degree position to about the 0-degree position and the lower plunger element 178 can be oriented 180-degrees from the location of the upper plunger element 178. The 0-degree position is understood as the top dead center position of a full circle when looking from an end view of the catheter hub in the distal direction.

Thus, when the various components are angled as shown in FIG. 15 and in accordance with aspects of the invention, any tilting of the needle 108 during retraction thereof following successful venipuncture, and therefore tilting of the tip protector 132, will simply cause the tip protector to slide against the inside surface 270 (FIG. 3B) of one or both of the plunger elements 178, 178. As the inside surfaces 270 of the two plunger elements 178, 178 do not have edges to catch against, the needle guard 132 can readily be retracted away from the valve opener without snagging the valve opener in the process. Thus, when the valve actuator and the needle guard are orientated as shown in FIG. 15, the components of the valve opener are configured to operate freely without the elbows of the needle guard snagging the edges of the stabilizers to thereby lead to failure.

Methods of making and of using the catheter assemblies and components thereof are within the scope of the present invention.

Although limited embodiments of catheter assemblies and their components have been specifically described and illustrated herein, many modifications and variations will be apparent to those skilled in the art. Accordingly, it is to be understood that the catheter assemblies and their components constructed according to principles of the disclosed device, system, and method may be embodied other than as specifically described herein. The disclosure is also defined in the following claims.

Needle assemblies, catheter assemblies, components of needle and catheter assemblies, and method for using and making catheter assemblies or needle assemblies in accordance with aspects of the invention may be covered by the following exemplary aspects:
Exemplary aspect 1: A needle assembly comprising: a needle hub with a needle having a needle tip with a needle bevel extending from a distal end of the needle hub; a catheter tube attached to a catheter hub having a body comprising an interior surface defining an interior cavity, the needle extending through the catheter tube and the needle tip out a distal end of the catheter tube in a ready to use position with the needle bevel oriented to a 0-degree position; a valve positioned in the interior cavity of the catheter hub and in contact with an internal shoulder of the catheter hub, said valve comprising an outer perimeter and a wall having at least one slit defining a plurality of flaps; a valve opener sized and shaped to open the plurality of slits to open the valve, said valve opener comprising a nose section having an activation end and a continuous perimeter section having a bore passing therethrough, two plunger elements, including a first plunger element and a second plunger element, each having two lengthwise edges extending proximally of the nose section, and two stabilizer elements; wherein each stabilizer element connects to the two plunger elements so that the two stabilizer elements and the two plunger elements define a stabilizing ring having a continuous surface and the stabilizing ring being spaced from the continuous perimeter section of the nose section by two through passages, which include a first through passage and a second through passage; a needle guard comprising a proximal wall and two resilient arms located in a holding space between the two stabilizer elements, wherein each resilient arm comprises an elbow and the needle guard has two resilient arms and two elbows, which include a first elbow and a second elbow; wherein (1) the first plunger element is oriented to about a 285-degree position to about the 0-degree position and the second plunger element is oriented 180-degrees from the first plunger element, or (2) the first plunger element is oriented to about a 1-degree position to about the 75-degree position and second plunger element is oriented 180-degrees from the first plunger element; and wherein the 0-degree position is a top dead center position of a full circle when looking from an end view of the catheter hub in a distal direction.
Exemplary aspect 2: The needle assembly according to exemplary aspect 1, wherein the first elbow is located in the first through passage and the second elbow is located in the second through passage in the ready to use position.
Exemplary aspect 3: The needle assembly according to any one of exemplary aspects 1 or 2, wherein the two arms of the needle guard bisect the two plunger elements.
Exemplary aspect 4: The needle assembly according to any one of exemplary aspects 1 to 3, wherein the valve comprises a proximally facing wall surface and wherein the activation end of the valve opener is urged against the proximally facing wall surface to push the plurality of flaps against the interior surface of the catheter hub in a used position.
Exemplary aspect 5: The needle assembly according to any one of exemplary aspects 1 to 4, wherein the plurality of slits define three slits defining three flaps.
Exemplary aspect 6: The needle assembly according to any one of exemplary aspects 1 to 5, wherein the first elbow is in contact with a perimeter of the first through passage.
Exemplary aspect 7: The needle assembly according to any one of exemplary aspects 1 to 6, wherein the first elbow is in contact with the interior surface of the catheter hub.
Exemplary aspect 8: The needle assembly according to any one of exemplary aspects 1 to 7, wherein the first elbow is in contact with a perimeter of the first through passage and the interior surface of the catheter hub.
Exemplary aspect 9: The needle assembly according to any one of exemplary aspects 1 to 8, wherein the first elbow is in contact with an edge of the stabilizing ring.
Exemplary aspect 10: The needle assembly according to any one of exemplary aspects 1 to 9, wherein the valve comprises a valve disc and a valve skirt extending in a proximal direction of the valve disc and defining a valve cavity.
Exemplary aspect 11: The needle assembly according to any one of exemplary aspects 1 to 10, wherein the second elbow is in contact with the interior surface of the catheter hub.
Exemplary aspect 12: The needle assembly according to any one of exemplary aspects 1 to 11, wherein the nose section has a frustoconical shape.
Exemplary aspect 13: The needle assembly according to any one of exemplary aspects 1 to 12, wherein the second elbow is in contact with a perimeter of the second through passage.
Exemplary aspect 14: The needle assembly according to any one of exemplary aspects 1 to 13, wherein the valve opener comprises a projection extending radially of a lengthwise axis of the valve opener, and wherein the interior surface of the catheter hub comprises a shoulder for limiting proximal movement of the projection.
Exemplary aspect 15: The needle assembly according to any one of exemplary aspects 1 to 14, wherein the projection is a first projection and wherein the valve opener comprises a second projection spaced from the first projection.
Exemplary aspect 16: The needle assembly according to any one of exemplary aspects 1 to 15, wherein the valve opener comprises a projection extending radially of a lengthwise axis on an exterior surface of one of the two plunger elements.
Exemplary aspect 17: The needle assembly according to any one of exemplary aspects 1 to 16, further comprising a skirt section extending distally of the wall.
Exemplary aspect 18: The needle assembly according to any one of exemplary aspects 1 to 17, wherein the first stabilizer element has a distal edge and the second stabilizer element has a distal edge.
Exemplary aspect 19: The needle assembly according to any one of exemplary aspects 1 to 18, wherein the distal edge of the first stabilizer element is parallel to the distal edge of the second stabilizing element.
Exemplary aspect 20: The needle assembly according to any one of exemplary aspects 1 to 19, wherein the distal edge of the first stabilizer element is angled to the distal edge of the second stabilizing element.
Exemplary aspect 21: The needle assembly according to any one of exemplary aspects 1 to 20, further comprising a projection on each of the two plunger elements to define two projections, wherein the two projections are located distally of the stabilizing ring.
Exemplary aspect 22: The needle assembly according to any one of exemplary aspects 1 to 21, wherein the needle guard, the valve, and the valve opener are entirely located within the interior cavity of the catheter hub.
Exemplary aspect 23: The needle assembly according to any one of exemplary aspects 1 to 22, further comprising a protective cap having a sleeve located around the catheter tube and the needle.
Exemplary aspect 24: The needle assembly according to any one of exemplary aspects 1 to 23, further comprising a bushing wedging a proximal end of the catheter tube against the interior surface of the catheter hub and a resilient element located inside the catheter hub between the bushing and the valve disc, and wherein the resilient element is sized and shaped to push the valve disc in a proximal direction.
Exemplary aspect 25: The needle assembly according to any one of exemplary aspects 1 to 24, wherein the resilient element is an elastic element or a spring.
Exemplary aspect 26: The needle assembly according to any one of exemplary aspects 1 to 25, wherein the wall of the valve has a generally flat or smooth distally facing wall surface.
Exemplary aspect 27: The needle assembly according to any one of exemplary aspects 1 to 26, further comprising a projection on each of the two plunger elements to define two projections, wherein the two projections are located distally of the stabilizing ring.
Exemplary aspect 28: The needle assembly according to any one of exemplary aspects 1 to 27, wherein the first plunger element is oriented to about the 285-degree position to about the 0-degree position but not at a 270-degree position or a 90-degree position.
Exemplary aspect 29: The needle assembly according to any one of exemplary aspects 1 to 28, wherein the angular position is relative to a mid-point of a width of the first plunger element and a mid-point of a width the second plunger element.
Exemplary aspect 30: The needle assembly according to any one of exemplary aspects 1 to 29, wherein each of the two resilient arms of the needle guard has a smooth or flat profile between an elongated arm portion and the elbow, with only a single bend or change in direction.
Exemplary aspect 31: The needle assembly according to any one of exemplary aspects 1 to 30, wherein each of the two resilient arms of the needle guard includes a cut-out having a lip that resembles a hook.
Exemplary aspect 32: The needle assembly according to any one of exemplary aspects 1 to 31, wherein each cut-out straddles the needle to provide lateral stability.
Exemplary aspect 33: The needle assembly according to any one of exemplary aspects 1 to 32, wherein a curved lip on a first arm of the two resilient arms has a straight edge.
Exemplary aspect 34: The needle assembly according to any one of exemplary aspects 1 to 33, wherein a curved lip on a second arm of the two resilient arms having a cut-out resembling an arc.
Exemplary aspect 35: The needle assembly according to any one of exemplary aspects 1 to 34, wherein the cut-out on the curved lip of the second arm changes a location where a surface of the curved lip contacts the needle.
Exemplary aspect 36: The needle assembly according to any one of exemplary aspects 1 to 35, wherein ribs are disposed on the two resilient arms.
Exemplary aspect 37: The needle assembly according to any one of exemplary aspects 1 to 36, wherein tabs extend from edges of a distal wall of each of the two resilient arms.
Exemplary aspect 38: The needle assembly according to any one of exemplary aspects 1 to 37, wherein the tabs limit sideway pivoting of a needle tip once the needle tip is captured within the needle guard.
Exemplary aspect 39: A method of assembling a needle assembly comprising: providing a catheter hub with a catheter tube having an open distal end and a proximal end attached to the catheter hub by a bushing, said catheter hub comprising a hub body having an interior surface defining an interior cavity and a proximal opening; positioning a valve comprising an outer perimeter and a wall having three slits defining three flaps in the interior cavity and proximal of the bushing; positioning a valve opener inside the interior cavity of the catheter hub to slidably push open the valve when actuated by a male medical implement, said valve opener comprising a nose section having an activation end and a continuous perimeter section having a bore passing therethrough, two plunger elements, including a first plunger element and a second plunger element, each having two lengthwise edges extending proximally of the nose section, and two stabilizer elements; wherein each stabilizer element connects to the two plunger elements so that the two stabilizer elements and the two plunger elements define a stabilizing ring having a continuous surface and the stabilizing ring being spaced from the continuous perimeter section of the nose section by two through passages, which include a first through passage and a second through passage; placing a needle guard comprising a proximal wall and two resilient arms in a holding space between the two stabilizer elements, wherein each resilient arm comprises an elbow and the needle guard has two resilient arms and two elbows, which include a first elbow and a second elbow; placing a needle, which is attached to a needle hub, through the catheter hub, the valve, and the catheter tube so that a tip of the needle with a needle bevel extends out the open distal end of the catheter tube in a ready to use position; orienting (1) the first plunger element to about a 285-degree position to about the 0-degree position and the second plunger element 180-degrees from the first plunger element, or (2) the first plunger element to about a 1-degree position to about the 75-degree position and second plunger element 180-degrees from the first plunger element; and wherein the 0-degree position is a top dead center position of a full circle when looking from an end view of the catheter hub in a distal direction.
Exemplary aspect 40: The method of assembly according to exemplary aspect 39, further comprising orienting the first elbow to a 90-degree position and orienting the second elbow to a 270-degree position.
Exemplary aspect 41: The method of assembly according to any one of exemplary aspects 39 or 40, wherein the valve comprises a proximally facing wall surface and wherein the activation end of the valve opener is configured to urge against the proximally facing wall surface to push the three flaps against the interior surface of the catheter hub in a used position.
Exemplary aspect 42: The method of assembly according to any one of exemplary aspects 39 to 41, wherein the first elbow is placed in contact with a perimeter of the first through passage.
Exemplary aspect 43: The method of assembly according to any one of exemplary aspects 39 to 42, wherein the first elbow is placed in contact with the interior surface of the catheter hub.
Exemplary aspect 44: The method of assembly according to any one of exemplary aspects 39 to 43, wherein the first elbow is placed in contact with a perimeter of the first through passage and the interior surface of the catheter hub.
Exemplary aspect 45: The method of assembly according to any one of exemplary aspects 39 to 44, wherein the first elbow is moved into contact with an edge of the stabilizing ring.
Exemplary aspect 46: The method of assembly according to any one of exemplary aspects 39 to 45, wherein the valve comprises a valve disc and a valve skirt extending in a proximal direction of the valve disc and defining a valve cavity.
Exemplary aspect 47: The method of assembly according to any one of exemplary aspects 39 to 46, wherein the activation end is placed within the valve cavity in the ready to use position.
Exemplary aspect 48: The method of assembly according to any one of exemplary aspects 39 to 47, wherein the second elbow is in contact with the interior surface of the catheter hub.
Exemplary aspect 49: The method of assembly according to any one of exemplary aspects 39 to 48, wherein the nose section has a frustoconical shape.
Exemplary aspect 50: The method of assembly according to any one of exemplary aspects 39 to 49, wherein the second elbow is in contact with a perimeter of the second through passage.
Exemplary aspect 51: A method for minimizing snagging of a valve opener comprising rotating angular positions of first and second plunger elements.
Exemplary aspect 52: The method for minimizing according to exemplary aspect 51 further comprising rotating angular positions of first and second resilient arms of a needle guard.
Exemplary aspect 53: The method for minimizing according to any one of exemplary aspects 51 or 52, wherein each of the two resilient arms of the needle guard has a smooth or flat profile between an elongated arm portion and the elbow, with only a single bend or change in direction to minimize hitting stabilizers on the valve opener with the elbow.
Exemplary aspect 54: Additional method for minimizing as disclosed in the context of rotating the valve opening and preventing the needle guard from snagging the one or more stabilizers of the valve opener when retracting the needle guard following successful venipuncture.

What is claimed is:

1. A needle assembly comprising:
a needle hub with a needle having a needle tip with a needle bevel extending from a distal end of the needle hub;
a catheter tube attached to a catheter hub having a body comprising an interior surface defining an interior cavity, the needle extending through the catheter tube and the needle tip out a distal end of the catheter tube in a ready to use position with the needle bevel oriented to a 0-degree position;
a valve positioned in the interior cavity of the catheter hub and in contact with an internal shoulder of the catheter hub, said valve comprising an outer perimeter and a wall having at least one slit defining a plurality of flaps;
a valve opener sized and shaped to open the plurality of slits to open the valve, said valve opener comprising a nose section having an activation end and a continuous perimeter section having a bore passing therethrough, two plunger elements, including a first plunger element and a second plunger element, each having two lengthwise edges extending proximally of the nose section, and two stabilizer elements; wherein each stabilizer element connects to the two plunger elements so that the two stabilizer elements and the two plunger elements define a stabilizing ring having a continuous surface and the stabilizing ring being spaced from the continuous perimeter section of the nose section by two through passages, which include a first through passage and a second through passage;
a needle guard comprising a proximal wall and two resilient arms located in a holding space between the two stabilizer elements, wherein each resilient arm comprises an elbow and the needle guard has two resilient arms and two elbows, which include a first elbow and a second elbow;
wherein (1) the first plunger element is oriented to about a 285-degree position to about the 0-degree position and the second plunger element is oriented 180-degrees from the first plunger element, or (2) the first plunger element is oriented to about a 1-degree position to about the 75-degree position and second plunger element is oriented 180-degrees from the first plunger element; and
wherein the 0-degree position is a top dead center position of a full circle when looking from an end view of the catheter hub in a distal direction.

2. The needle assembly of claim 1, wherein the first elbow is located in the first through passage and the second elbow is located in the second through passage in the ready to use position.

3. The needle assembly of claim 1, wherein the two arms of the needle guard bisect the two plunger elements.

4. The needle assembly of claim 1, wherein the valve comprises a proximally facing wall surface and wherein the activation end of the valve opener is urged against the proximally facing wall surface to push the plurality of flaps against the interior surface of the catheter hub in a used position.

5. The needle assembly of claim 1, wherein the first elbow is in contact with a perimeter of the first through passage.

6. The needle assembly of claim 1, wherein the first elbow is in contact with the interior surface of the catheter hub.

7. The needle assembly of claim 1, wherein the first elbow is in contact with a perimeter of the first through passage and the interior surface of the catheter hub.

8. The needle assembly of claim 1, wherein the first elbow is in contact with an edge of the stabilizing ring.

9. The needle assembly of claim 1, wherein the valve comprises a valve disc and a valve skirt extending in a proximal direction of the valve disc and defining a valve cavity.

10. The needle assembly of claim 1, wherein the valve opener comprises a projection extending radially of a lengthwise axis of the valve opener, and wherein the interior surface of the catheter hub comprises a shoulder for limiting proximal movement of the projection.

11. The needle assembly of claim 1, wherein the distal edge of the first stabilizer element is parallel to the distal edge of the second stabilizing element.

12. The needle assembly of claim 1, wherein the distal edge of the first stabilizer element is angled to the distal edge of the second stabilizing element.

13. The needle assembly of claim 1, further comprising a bushing wedging a proximal end of the catheter tube against the interior surface of the catheter hub and a resilient element located inside the catheter hub between the bushing and the valve disc, and wherein the resilient element is sized and shaped to push the valve disc in a proximal direction.

14. The needle assembly of claim 13, wherein the resilient element is an elastic element or a spring.

15. A method of assembling a needle assembly comprising:
providing a catheter hub with a catheter tube having an open distal end and a proximal end attached to the catheter hub by a bushing, said catheter hub comprising a hub body having an interior surface defining an interior cavity and a proximal opening;
positioning a valve comprising an outer perimeter and a wall having three slits defining three flaps in the interior cavity and proximal of the bushing;
positioning a valve opener inside the interior cavity of the catheter hub to slidably push open the valve when actuated by a male medical implement, said valve opener comprising a nose section having an activation end and a continuous perimeter section having a bore passing therethrough, two plunger elements, including a first plunger element and a second plunger element, each having two lengthwise edges extending proximally of the nose section, and two stabilizer elements; wherein each stabilizer element connects to the two plunger elements so that the two stabilizer elements and the two plunger elements define a stabilizing ring having a continuous surface and the stabilizing ring being spaced from the continuous perimeter section of the nose section by two through passages, which include a first through passage and a second through passage;
placing a needle guard comprising a proximal wall and two resilient arms in a holding space between the two stabilizer elements, wherein each resilient arm comprises an elbow and the needle guard has two resilient arms and two elbows, which include a first elbow and a second elbow;
placing a needle, which is attached to a needle hub, through the catheter hub, the valve, and the catheter tube so that a tip of the needle with a needle bevel extends out the open distal end of the catheter tube in a ready to use position;
orienting (1) the first plunger element to about a 285-degree position to about the 0-degree position and the second plunger element 180-degrees from the first plunger element, or (2) the first plunger element to about a 1-degree position to about the 75-degree position and second plunger element 180-degrees from the first plunger element; and
wherein the 0-degree position is a top dead center position of a full circle when looking from an end view of the catheter hub in a distal direction.

16. The needle assembly of claim 2, wherein the first elbow is in contact with a perimeter of the first through passage.

17. The needle assembly of claim 3, wherein the first elbow is in contact with the interior surface of the catheter hub.

18. The needle assembly of claim 2, wherein the first elbow is in contact with a perimeter of the first through passage and the interior surface of the catheter hub.

19. The needle assembly of claim 3, wherein the first elbow is in contact with an edge of the stabilizing ring.

20. The needle assembly of claim 5, wherein the distal edge of the first stabilizer element is angled to the distal edge of the second stabilizing element.
